(19) United States
(12) Patent Application Publication
(10) Pub. No.: US 2013/0345243 A1
(43) Pub. Date: Dec. 26, 2013

(54) 1H-PYROLLO[3,2-D]PYRIMIDINEDIONE DERIVATIVES

(71) Applicants: Anne Marie Jeanne Bouillot, Les Ulis (FR); Alain Claude-Marie Daugan, Les Ulis (FR); Yann Lamotte, Les Ulis (FR); Olivier Mirguet, Les Ulis (FR)

(72) Inventors: Anne Marie Jeanne Bouillot, Les Ulis (FR); Alain Claude-Marie Daugan, Les Ulis (FR); Yann Lamotte, Les Ulis (FR); Olivier Mirguet, Les Ulis (FR)

(73) Assignee: GLAXOSMITHKLINE LLC, Philadelphia, PA (US)

(21) Appl. No.: 14/003,433

(22) PCT Filed: Mar. 5, 2012
(86) PCT No.: PCT/EP2012/053732

§ 371 Date: Sep. 5, 2013

Related U.S. Application Data
(60) Provisional application No. 61/449,857, filed on Mar. 7, 2011.

(51) Int. Cl. C07D 487/04

(52) U.S. Cl. CPC C07D 487/04
USPC 514/265.1; 544/280

(57) ABSTRACT

The present invention relates to pyrimidinedione compounds of formula (I),

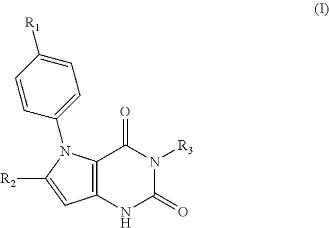

salts thereof, to pharmaceutical compositions containing them and their use in medicine. In particular, the invention relates to compounds of formula (I) or salts thereof as activators of AMPK.

### FIELD OF THE INVENTION

The present invention relates to a novel class of compounds which are activators of AMP-activated protein kinase (AMPK) (AMPK-activators), compositions comprising said compounds, methods of synthesis and uses for such compounds in treating various diseases mediated by AMPK, such as type 1 (Type I) diabetes, type 2 (Type II) diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, cardiac ischemia, virus infection (HIV, cytomegalovirus and hepatitis C) or cancer.

### BACKGROUND OF THE INVENTION

AMPK has been established as a sensor and regulator of cellular energy homeostasis (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003)). Allosteric activation of this kinase due to rising AMP levels occurs in states of cellular energy depletion. The resulting serine/threonine phosphorylation of target enzymes leads to an adaptation of cellular metabolism to the low energy state. The net effect of AMPK activation induced changes is inhibition of ATP consuming processes and activation of ATP generating pathways, and therefore regeneration of ATP stores. Examples of AMPK substrates include acetyl-CoA-carboxylase (ACC) and HMG-CoA-reductase (Carling, D. et. al. A common bicyclic protein kinase cascade inactivates the regulatory enzymes of fatty acid and cholesterol biosynthesis. FEBS Letters 223:217 (1987)). Phosphorylation and therefore inhibition of ACC leads to a decrease in fatty acid synthesis (ATP-consuming) and at the same time to an increase in fatty acid oxidation (ATP-generating). Phosphorylation and resulting inhibition of HMG-CoA reductase leads to a decrease in cholesterol synthesis. Other substrates of AMPK include hormone sensitive lipase (Garton, A. J. et. al. Phosphorylation of bovine hormone-sensitive lipase by the AMP-activated protein kinase. A possible antilipolytic mechanism. Eur. J. Biochem. 179:249 (1989)), glycerol-3-phosphate acyltransferase (Muoio, D. M. et. al. AMP-activated kinase reciprocally regulates triacylglycerol synthesis and fatty acid oxidation in liver and muscle: evidence that sn-glycerol-3-phosphate acyltransferase is a novel target. Biochem. J. 338:783 (1999)), malonyl-CoA decarboxylase (Saha, A. K. et. al. Activation of malonyl-CoA decarboxylase in rat skeletal muscle by contraction and the AMP-activated protein kinase activator 5-aminoimidazole-4-carboxamide-1-.beta.-D-ribofuranoside. J. Biol. Chem. 275:24279 (2000)), some of which are potential drug targets for components of metabolic syndrome. Additional processes that are believed to be regulated through AMPK activation, but for which the exact AMPK substrates have not been identified, include stimulation of glucose transport in skeletal muscle and expressional regulation of key genes in fatty acid and glucose metabolism in liver (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003), Musi, N. and Goodyear, L. J. Targeting the AMP-activated protein kinase for the treatment of Type 2 diabetes. Current Drug Targets-Immune, Endocrine and Metabolic Disorders 2:119 (2002)). For example, decreased expression of glucose-6-phosphatase (Lochhead, P. A. et. al. 5-aminoimidazole-4-carboxamide riboside mimics the effects of insulin on the expression of the 2 key gluconeogenic genes PEPCK and glucose-6-phosphatase. Diabetes 49:896 (2000)), a key enzyme in hepatic glucose production, and SREBP-1c (Zhou, G. et. al. Role of AMP-activated protein kinase in mechanism of metformin action. The J. of Clin. Invest. 108: 1167 (2001)), a key lipogenic transcription factor, has been found following AMPK stimulation.

More recently an involvement of AMPK in the regulation of not only cellular but also whole body energy metabolism has become apparent. It was shown that the adipocyte-derived hormone leptin leads to a stimulation of AMPK and therefore to an increase in fatty acid oxidation in skeletal muscle (Minokoshi, Y. et. al. Leptin stimulates fatty-acid oxidation by activating AMP-activated protein kinase. Nature 415: 339 (2002)). Adiponectin, another adipocyte derived hormone leading to improved carbohydrate and lipid metabolism, has been demonstrated to stimulate AMPK in liver and skeletal muscle (Yamauchi, T. et. al. Adiponectin stimulates glucose utilization and fatty acid oxidation by activating AMP-activated protein kinase. Nature Medicine 8: 1288 (2002), Tomas, E. et. al. Enhanced muscle fat oxidation and glucose transport by ACRP30 globular domain: Acetyl-CoA carboxylase inhibition and AMP-activated protein kinase activation. PNAS 99: 16309 (2002)). The activation of AMPK in these circumstances seems to be independent of increasing cellular AMP levels but rather due to phosphorylation by one or more yet to be identified upstream kinases.

Based on the knowledge of the above-mentioned consequences of AMPK activation, certain beneficial effects could be expected from in vivo activation of AMPK. In liver, decreased expression of gluconeogenic enzymes could reduce hepatic glucose output and improve overall glucose homeostasis, and both direct inhibition and/or reduced expression of key enzymes in lipid metabolism could lead to decreased fatty acid and cholesterol synthesis and increased fatty acid oxidation. Stimulation of AMPK in skeletal muscle could increase glucose uptake and fatty acid oxidation with resulting improvement of glucose homeostasis and, due to a reduction in intra-myocyte triglyceride accumulation, to improved insulin action. Finally, the increase in energy expenditure could lead to a decrease in body weight. The combination of these effects in metabolic syndrome could be expected to reduce the risk for acquiring cardiovascular diseases.

Several studies in rodents support this hypothesis (Bergeron, R. et. al. Effect of 5-aminoimidazole-4-carboxamide-1(beta)-D-ribofuranoside infusion on in vivo glucose metabolism in lean and obese Zucker rats. Diabetes 50:1076 (2001), Song, S. M. et. al. 5-Aminoimidazole-4-darboxamide ribonucleoside treatment improves glucose homeostasis in insulin-resistant diabeted (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying feature of the insulin resistance syndrome. Diabetes 51: 2199 (2002)). Until recently most in vivo studies have relied on the AMPK activator AICAR, a cell permeable precursor of ZMP. ZMP acts as an intracellular AMP mimic, and, when accumulated to high enough levels, is able to stimulate AMPK activity (Corton, J. M. et. al. 5-Aminoimidazole-4-carboxamide ribonucleoside, a specific method for activating AMP-activated protein kinase in intact cells? Eur. J. Biochem. 229: 558 (1995)). However, ZMP also acts as an AMP mimic in the regulation of other enzymes, and is therefore not a specific AMPK activator (Musi, N. and Goodyear, L. J. Targeting the AMP-activated protein kinase for the treatment of Type 2 diabetes. Current Drug Targets-Immune, Endocrine and Metabolic Disorders 2:119 (2002)). Several in vivo studies have demonstrated beneficial effects of both acute and chronic AICAR administration in rodent models of obesity and Type 2 diabetes (Bergeron, R. et. al. Effect of 5-aminoimidazole-4-carboxamide-1(beta)-D-ribofuranoside infusion on in vivo glucose metabolism in lean and obese Zucker rats. Diabetes 50:1076 (2001), Song, S. M. et. al. 5-Aminoimidazole-4-darboxamide ribonucleoside treatment improves glucose homeostasis in insulin-resistant diabetic (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying feature of the insulin resistance syndrome. Diabetes 51: 2199 (2002)). For example, 7 week AICAR administration in the obese Zucker (fa/fa) rat leads to a reduction in plasma triglycerides and free fatty acids, an increase in HDL cholesterol, and a normalization of glucose metabolism as assessed by an oral glucose tolerance test (Minokoshi, Y. et. al. Leptin stimulates fatty-acid oxidation by activating AMP-activated protein kinase. Nature 415: 339 (2002)). In both ob/ob and db/db mice, 8 day AICAR administration reduces blood glucose by 35% (Halseth, A. E. et. al. Acute and chronic treatment of ob/ob and db/db mice with AICAR decreases blood glucose concentrations. Biochem. and Biophys. Res. Comm. 294:798 (2002)). In addition to AICAR, more recently it was found that the diabetes drug metformin can activate AMPK in vivo at high concentrations (Zhou, G. et. al. Role of AMP-activated protein kinase in mechanism of metformin action. The J. of Clin. Invest. 108: 1167 (2001), Musi, N. et. al. Metformin increases AMP-activated protein kinase activity in skeletal muscle of subjects with Type 2 diabetes. Diabetes 51: 2074 (2002)), although it has to be determined to what extent its antidiabetic action relies on this activation. As with leptin and adiponectin, the stimulatory effect of metformin is indirect via a mild inhibition of mitochondrial respiratory chain complex 1 (Leverve X. M. et al. Mitochondrial metabolism and type-2 diabetes: a specific target of metformin. Diabetes Metab. 29: 6588 (2003)). In addition to pharmacologic intervention, several transgenic mouse models have been developed in the last years and initial results are becoming available. Expression of dominant negative AMPK in skeletal muscle of transgenic mice has demonstrated that the AICAR effect on stimulation of glucose transport is dependent on AMPK activation (Mu, J. et. al. A role for AMP-activated protein kinase in contraction and hypoxia-regulated glucose transport in skeletal muscle. Molecular Cell 7: 1085 (2001)), and therefore likely not caused by non-specific ZMP effects. Similar studies in other tissues will help to further define the consequences of AMPK activation. It is believed that pharmacologic activation of AMPK may have benefits in relation to metabolic syndrome with improved glucose and lipid metabolism and a reduction in body weight. To qualify a patient as having metabolic syndrome, three out of the five following criteria must be met: elevated blood pressure above 130/85 mmHg, fasting blood glucose above 110 mg/dl, abdominal obesity above 40″ (men) or 35″ (women) waist circumference, and blood lipid changes as defined by an increase in triglycerides above 150 mg/dl or decreased HDL cholesterol below 40 mg/dl (men) or 50 mg/dl (women). Therefore, the combined effects that may be achieved through activation of AMPK in a patient who qualifies as having metabolic syndrome would raise the interest of this target.

Lowering of blood pressure has been reported to be a consequence of AMPK activation (Buhl, E. S. et. al. Long-term AICAR administration reduces metabolic disturbances and lowers blood pressure in rats displaying feature of the insulin resistance syndrome. Diabetes 51: 2199 (2002)), therefore activation of AMPK might have beneficial effects in hypertension. Through combination of some or all of the above-mentioned effects stimulation of AMPK may to reduce the incidence of cardiovascular diseases (e.g. MI, stroke). Increased fatty acid synthesis is a characteristic of many tumor cells, therefore decreased synthesis of fatty acids through activation of AMPK could be useful as a cancer therapy (Huang X. et al. Important role of the LKB1-AMPK pathway in suppressing tumorigenesis in PTEN-deficient mice. Biochem J. 412: 211 (2008). AMPK can also be considered as a metabolic tumor suppressor and AMPK activators could be helpful in general cancer therapy (Luo Z. Et al. AMPK as a metabolic tumor suppressor: control of metabolism and cell growth. Future Oncol. 6: 457 (2010)). Pharmacological activation of the LKB1/AMPK/mTOR axis using known AMPK activators such as metformin, AICAR or A-769662 induce in most studies a dramatic suppression of cancer cell growth, demonstrating that the reinforcement of the tumor suppressive functions of LKB1/AMPK is a valuable therapeutic strategy for both solid tumors and hematological cancers such as AML, CML (Green A. S. et al. LKB1/AMPK/mTOR signaling pathway in hematological malignancies: From metabolism to cancer cell biology. Cell Cycle 10: 2115 (2011). Micic D. et al. Metformin: Its emerging role in oncology. Hormones 10:5 (2011)). The connection of AMPK with several tumour suppressors suggests that therapeutic manipulation of this pathway using AMPK activators warrants further investigation in patients with cancer such as Peutz-Jeghers syndrome, a dominantly inherited cancer-predisposition syndrome in which, at least 80% of all reported cases are caused by mutations that inactivate the gene encoding LKB1 (chromosome 19p13.3), AMPK upstream kinase (Shackelford D. B.; Shaw R. J. The LKB1-AMPK pathway: metabolism and growth control in tumour suppression. Nature Rev. Cancer 2009, 9: 563 (2009). Carling D. LKB1: a sweet side to Peutz-Jeghers syndrome? TRENDS in Molecular Medicine 12: 144 (2006)).

Stimulation of AMPK has been shown to stimulate production of ketone bodies from astrocytes (Blazquez, C. et. al. The AMP-activated protein kinase is involved in the regulation of ketone body production by astrocytes. J. Neurochem. 73: 1674 (1999)), and might therefore be a strategy to treat ischemic events in the brain. Stimulation of AMPK has been shown to improve cognition and neurodegenerative diseases in a mice model (Dagon Y. et al. Nutritional status, cognition, and survival: a new role for leptin and AMP kinase. J. Biol. Chem. 280:42142 (2005)). Stimulation of AMPK has been shown to stimulate expression of uncoupling protein 3 (UCP3) in skeletal muscle (Zhou, M. et. al. UCP-3 expression in skeletal muscle: effects of exercise, hypoxia, and AMP-activated protein kinase. Am. J. Physiol. Endocrinol. Metab. 279: E622 (2000)) and might therefore be a way to prevent damage from reactive oxygen species. Endothelial NO synthase (eNOS) has been shown to be activated through AMPK mediated phosphorylation (Chen, Z.-P., et. al. AMP-activated protein kinase phosphorylation of endothelial NO synthase. FEBS Letters 443: 285 (1999)), therefore AMPK activation may be used to improve local circulatory systems. AMPK has also been described to directly affect PGC-1alpha activity through phosphorylation and then regulate mitochondria biogenesis (Jager S, et al. AMP-activated protein kinase (AMPK) action in skeletal muscle via direct phosphorylation of PGC-1alpha. Proc Natl Acad Sci 104:12017 (2007)). AMPK activation can be then a way to treat mitochondrial disorders (e.g. sarcopenia and some mitochondrial rare diseases). Recently, several reports describe beneficial effect of AMPK activation on virus infection. While virus infection is found to reduce AMPK activity in infected cells or tissues, AMPK activation is proposed as a anti-viral therapy (Mankouri J. et al., Enhanced hepatitis C virus genome replication and lipid accumulation mediated by inhibition of AMP-activated protein kinase, Proc Natl Acad Sci 107: 11549 (2010)).

The use of AMPK activators may represent a strategy to protect the heart and other solid organs against cardiac ischemia as it has been demonstrated with A-769662 (Kim A. S. et al. A small molecule AMPK activator protects the heart against ischemia-reperfusion injury. J. Mol. Cell. Cardiology 51: 24 (2011)) or metformin (Yin M. et al. Metformin improves cardiac function in a non-diabetic rat model of 2 post-MI heart failure Am J Physiol Heart Circ Physiol 301: H459 (2011)).

### SUMMARY OF THE INVENTION

The present invention provides a compound of formula (I):

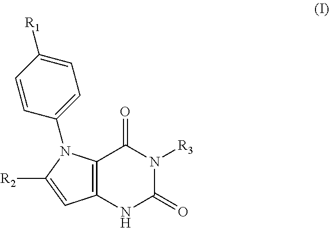

wherein
R¹ represents

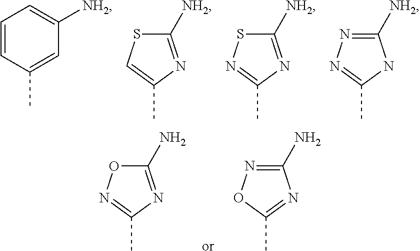

R² represents H, —C₁₋₄alkyl, CN, or halogen;
R³ represents
(a) —C₁₋₄alkyl substituted by one or two groups independently selected from: —OH and —CO₂H;
(b) —C₆₋₁₀aryl, or -(5-10 membered heteroaryl), wherein the —C₆₋₁₀aryl or -(5-10 membered heteroaryl) is optionally substituted by one or two groups independently selected from
   (i) —C₁₋₄alkyl wherein the alkyl group is unsubstituted or substituted by one or two groups independently selected from: —OH or —CO₂H,
   (ii) —OMe;
   (iii) —SMe
   (iv) —OH;
   (v) —CN;
   (vi) —NO₂;
   (vii) —CO₂H;
   (viii) —C₁₋₄alkylene(C═O)XC₁₋₄alkyl; and
   (ix) fluoro;
      X represents O or NR⁴; and
      R⁴ represents H or —C₁₋₄alkyl;
      or a salt thereof;
      provided that the compound of formula (I) is not 5-(4-(2-aminothiazol-4-yl)phenyl)-6-chloro-3-(3-fluoro-2-methylphenyl)-1H-pyrrolo[3,2-d]pyrimidine-2,4(3H,5H)-dione.

In another aspect, the present invention provides pharmaceutical compositions comprising a compound of formula (I) or a pharmaceutically acceptable salt thereof.

In another aspect, the present invention provides methods of treating type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, cardiac ischemia, virus infection (HIV, cytomegalovirus or hepatitis C) or cancer comprising administration of a therapeutically effective amount of a compound of formula (I) or a pharmaceutically acceptable salt thereof to a subject in need thereof.

In another aspect, the present invention provides methods of treating type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus or hepatitis C) or cancer comprising administration of a therapeutically effective amount of a compound of formula (I) or a pharmaceutically acceptable salt thereof to a subject in need thereof.

In another aspect, the present invention provides methods of treating diabetes, metabolic syndrome, atherosclerosis, dyslipidaemia, obesity, hypertension, cerebral ischemia, cognitive defect and cancer comprising administration of a therapeutically effective amount of a compound of formula (I) or a pharmaceutically acceptable salt thereof to a subject in need thereof.

In another aspect, the present invention provides methods of treating type 2 diabetes, obesity or dyslipidaemia comprising administration of a therapeutically effective amount of a compound of formula (I) or a pharmaceutically acceptable salt thereof to a subject in need thereof.

In another aspect, the present invention provides methods of treating cancer comprising administration of a therapeutically effective amount of a compound of formula (I) or a pharmaceutically acceptable salt thereof to a subject in need thereof.

In another aspect, the invention provides a compound of formula (I) or a pharmaceutically acceptable salt thereof for use in human or veterinary medical therapy.

In another aspect, the invention provides a compound of formula (I) or a pharmaceutically acceptable salt thereof, for use in the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, cardiac ischemia, virus infection (HIV, cytomegalovirus or hepatitis C) or cancer.

In another aspect, the invention provides a compound of formula (I) or a pharmaceutically acceptable salt thereof, for use in the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus or hepatitis C) or cancer.

In another aspect, the invention provides a compound of formula (I) or a pharmaceutically acceptable salt thereof, for use in the treatment of diabetes, metabolic syndrome, atherosclerosis, dyslipidaemia, obesity, hypertension, cerebral ischemia, cognitive defect or cancer.

In another aspect, the invention provides a compound of formula (I) or a pharmaceutically acceptable salt thereof, for use in the treatment of type 2 diabetes, obesity or dyslipidaemia.

In another aspect, the invention provides a compound of formula (I) or a pharmaceutically acceptable salt thereof, for use in the treatment of cancer.

In another aspect, the invention provides the use of a compound of formula (I) or a pharmaceutically acceptable salt thereof in the manufacture of a medicament for the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, cardiac ischemia, virus infection (HIV, cytomegalovirus or hepatitis C) or cancer.

In another aspect, the invention provides the use of a compound of formula (I) or a pharmaceutically acceptable salt thereof in the manufacture of a medicament for the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus or hepatitis C) or cancer.

In another aspect, the invention provides the use of a compound of formula (I) or a pharmaceutically acceptable salt thereof in the manufacture of a medicament for the treatment of diabetes, metabolic syndrome, atherosclerosis, dyslipidaemia, obesity, hypertension, cerebral ischemia, cognitive defect or cancer.

In another aspect, the invention provides the use of a compound of formula (I) or a pharmaceutically acceptable salt thereof in the manufacture of a medicament for the treatment of type 2 diabetes, obesity or dyslipidaemia.

In another aspect, the invention provides the use of a compound of formula (I) or a pharmaceutically acceptable salt thereof in the manufacture of a medicament for the treatment of cancer.

### DESCRIPTION OF THE EMBODIMENTS

All aspects and embodiments of the invention described herein are in respect of compounds of formula (I) or salts thereof, unless otherwise specified.

In one embodiment, R¹ represents

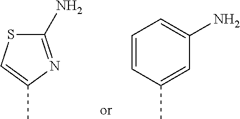

In a further embodiment, R¹ represents

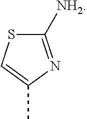

In an alternative further embodiment, R¹ represents

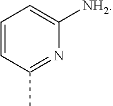

In one embodiment R² represents halogen or methyl. In one embodiment R² represents halogen. In a preferred embodiment, R² represents chloro.

In one embodiment, R³ represents
(a) —C₁₋₄alkyl substituted by one or two groups independently selected from: —OH and —CO₂H;
(b) C₆₋₁₀aryl optionally substituted with one or two groups independently selected from the group consisting of C₁₋₄alkyl, OMe, SMe, fluoro and CO₂H; or
(c) 5-10 membered heteroaryl optionally substituted with one or two groups independently selected from the group consisting of C₁₋₄alkyl, OMe, SMe, fluoro and CO₂H.

In a further embodiment, R³ represents C₆₋₁₀aryl optionally substituted with one or two groups independently selected from the group consisting of C₁₋₄alkyl, OMe, SMe, fluoro and CO₂H. In a still further embodiment, R³ represents phenyl, optionally substituted with one or two groups independently selected from the group consisting of C₁₋₄alkyl, OMe, SMe, fluoro and CO₂H. In a still further embodiment, R³ represents phenyl, optionally substituted with one or two groups independently selected from the group consisting of methyl, OMe, SMe, fluoro and CO₂H. In a still further embodiment, R³ is selected from the group consisting of 3-methoxyphenyl, 3-methylthiophenyl, 3-carboxyphenyl, 2-fluorophenyl, 2-methyl-4-methoxyphenyl, 4-methoxyphenyl, 3,5-dimethoxyphenyl, 2,5-dimethoxyphenyl, 2-methyl-3-methoxyphenyl and 2-methoxyphenyl.

In an alternative embodiment, R³ is C₁₋₄alkyl substituted by one or two groups independently selected from OH and CO₂H. In an further embodiment, R³ is C₁₋₄alkyl substituted with CO₂H. In a still further embodiment, R³ is CH₂CH₂CO₂H.

In an alternative embodiment, R³ is 5-10 membered heteroaryl, optionally substituted with one or two groups independently selected from the group consisting of C₁₋₄alkyl, OMe, SMe, fluoro and CO₂H. In a further embodiment, R³ is unsubstituted 5-10 membered heteroaryl. In a further embodiment, R³ is unsubstituted pyridyl or benzo[d][1,3]dioxol-5-yl. In a still further embodiment, R³ is unsubstituted 3-pyridyl. In a still further alternative embodiment, R³ is unsubstituted benzo[d][1,3]dioxol-5-yl.

Each of the aspects of the invention are independent unless stated otherwise. Nevertheless the skilled person will understand that all the permutations of the aspects herein described are within the scope of the invention. Thus it is to be understood that the present invention covers all combinations of suitable, convenient and exemplified aspects described herein.

As used herein, the term “alkyl” refers to a straight or branched saturated hydrocarbon chain containing the specified number of carbon atoms. For example, —C₁₋₄alkyl refers to a straight or branched alkyl containing at least 1, and at most 4, carbon atoms. Examples of “alkyl” as used herein include, but are not limited to, methyl, ethyl, n-propyl, n-butyl, isobutyl, isopropyl and t-butyl.

As used herein, the term “—C₆₋₁₀aryl” refers to a carbocyclic moiety containing 6 to 10 carbon ring-atoms. The term includes both monocyclic and bicyclic ring systems and bicyclic structures at least a portion of which is aromatic and the other part is saturated, partially or fully unsaturated. Examples of aryl groups as used herein include, but are not limited to, naphthyl, anthryl, phenanthryl, indanyl, indenyl, azulenyl, azulanyl and fluorenyl; phenyl and naphthyl; and more specifically phenyl.

As used herein, the term “halogen” or “halo” refers to a fluorine (fluoro), chlorine (chloro), bromine (bromo) or iodine (iodo) atom.

As used herein, the term “-(5-10 membered heteroaryl)” refers to an cyclic group containing 5 to 10 ring-atoms 1, 2, 3 or 4 of which are hetero-atoms independently selected from nitrogen, oxygen and sulphur and the remaining ring-atoms are carbon. This term includes both aromatic monocyclic and bicyclic ring systems and bicyclic structures at least a portion of which is aromatic and the other part is saturated, partially or fully unsaturated. Examples of “-(5-10 membered heteroaryl)” groups used herein include, but are not limited to, benzo[d][1,3]dioxolane and pyridine.

As used herein, the term “alkylene” refers to straight or branched chain saturated hydrocarbon linker groups containing the specified number of carbon atoms. For example, —C₁₋₄alkylene refers to a straight or branched “alkylene” containing at least 1, and at most 4, carbon atoms. Examples of “alkylene” as used herein include, but are not limited to, methylene (—CH₂—) and ethylene (—CH₂CH₂—).

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

Also included in the present invention are pharmaceutically acceptable salt complexes. In certain embodiments of the invention, pharmaceutically acceptable salts of the compounds according to formula I may be preferred over the respective free base or free acid because such salts impart greater stability or solubility to the molecule thereby facilitating formulation into a dosage form. Therefore, the present invention also covers the pharmaceutically acceptable salts of the compounds of formula (I).

Therefore, in one aspect of the invention there is provided a compound of formula (I) or a salt thereof wherein the salt is a pharmaceutically acceptable salt.

As used herein, the term “pharmaceutically acceptable”, refers to salts, molecular entities and other ingredients of compositions that are generally physiologically tolerable and do not typically produce untoward reactions when administered to a subject (e.g. human). The term “pharmaceutically acceptable” also means approved by a regulatory agency of the Federal or a state government or listed in the U.S. Pharmacopoeia or other generally recognized pharmacopoeia for use in a subject, and more particularly in humans.

As used herein, the term “subject” refers to an animal, in particular a mammal and more particularly to a human or a domestic animal or an animal serving as a model for a disease (e.g., mouse, monkey, etc.). In one aspect, the subject is a human.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

Suitable pharmaceutically acceptable salts will be apparent to those skilled in the art and include for example (where possible) base addition salts e.g. ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine or for example acid addition salts formed from acids which form non-toxic salts e.g. hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-toluensulphonate, methanesulphonic, ethanesulphonic, p-toluenesulphonic, and isethionate. For a review on suitable salts see Berge et al. J. Pharm. Sci., 1977, 66, 1-19. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”.

As used herein, the term “solvate” refers to a complex of variable stoichiometry formed by a solute (such as a compound of formula (I) or a salt thereof) and a solvent. Examples of suitable solvents include, but are not limited to, water, methanol, ethanol and acetic acid. Preferably the solvent used is a pharmaceutically acceptable solvent. Most preferably the solvent used is water and the solvate may also be referred to as a hydrate.

Solvates of compounds of formula (I) which are suitable for use in medicine are those wherein the solvent is pharmaceutically acceptable. However, solvates having non-pharmaceutically acceptable solvents may be useful as intermediates in the preparation of other compounds of formula (I) and their pharmaceutically acceptable salts.

As used herein, the term “compounds of the invention” means the compounds according to formula (I) and pharmaceutically acceptable salts thereof. The term “a compound of the invention” means any one of the compounds of the invention as defined below.

Prodrugs of the compounds of formula (I) are included within the scope of the present invention. In one embodiment, the compounds of formula (I) or salts thereof are not prodrugs.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987 and in D. Fleishner, S. Ramon and H. Barba “Improved oral drug delivery: solubility limitations overcome by the use of prodrugs”, Advanced Drug Delivery Reviews (1996) 19(2) 115-130. Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxy, amine or carboxylic acid groups are bonded to any group that, when administered to a patient, cleaves to form the hydroxy, amine or carboxylic acid groups. Thus, representative examples of prodrugs include (but are not limited to) phosphonate, carbamate, acetate, formate and benzoate derivatives of hydroxy, amine or carboxylic acid functional groups of the compounds of formula (I).

Certain compounds of formula (I) may exist in stereoisomeric forms (e.g. they may contain one or more asymmetric carbon atoms). The individual stereoisomers (enantiomers and diastereomers) and mixtures or racemic mixtures thereof are included within the scope of the present invention. The invention also extends to conformational isomers of compounds of formula (I). Likewise, it is understood that compounds of formula (I) may exist in tautomeric forms other than that shown in the formula and these are also included within the scope of the present invention.

It will be appreciated that racemic compounds of formula (I) may be optionally resolved into their individual enantiomers. Such resolutions may conveniently be accomplished by standard methods known in the art. For example, a racemic compound of formula (I) may be resolved by chiral preparative HPLC. An individual stereoisomer may also be prepared from a corresponding optically pure intermediate or by resolution, such as H.P.L.C. of the corresponding mixture using a suitable chiral support or by fractional crystallisation of the diastereoisomeric salts formed by reaction of the corresponding mixture with a suitable optically active acid or base, as appropriate.

In one aspect, the present invention comprises a compound of formula (I) selected from the group consisting of:
5-(4-(6-aminopyridin-2-yl)phenyl)-6-chloro-3-(3-methoxyphenyl)-1H-pyrrolo[3,2-d]pyrimidine-2,4(3H,5H)-dione;
5-[4-(2-amino-1,3-thiazol-4-yl)phenyl]-6-chloro-3-[3-(methyloxy)phenyl]-1H-pyrrolo[3,2-d]pyrimidine-2,4(3H,5H)-dione;
3-(5-(4-(2-aminothiazol-4-yl)phenyl)-6-chloro-2,4-dioxo-1H-pyrrolo[3,2-d]pyrimidin-3(2H,4H,5H)-yl)propanoic acid;
5-(4-(2-aminothiazol-4-yl)phenyl)-6-chloro-3-(3,5-dimethoxyphenyl)-1H-pyrrolo[3,2-d]pyrimidine-2,4(3H,5H)-dione;
5-(4-(2-aminothiazol-4-yl)phenyl)-3-(benzo[d][1,3]dioxol-5-yl)-6-chloro-1H-pyrrolo[3,2-d]pyrimidine-2,4(3H,5H)-dione;
5-(4-(2-aminothiazol-4-yl)phenyl)-6-chloro-3-(pyridin-3-yl)-1H-pyrrolo[3,2-d]pyrimidine-2,4(3H,5H)-dione;
5-(4-(2-aminothiazol-4-yl)phenyl)-6-chloro-3-(3-methoxy-2-methylphenyl)-1H-pyrrolo[3,2-d]pyrimidine-2,4(3H,5H)-dione;
5-(4-(2-aminothiazol-4-yl)phenyl)-6-chloro-3-(2-methoxyphenyl)-1H-pyrrolo[3,2-d]pyrimidine-2,4(3H,5H)-dione;
3-(5-(4-(2-aminothiazol-4-yl)phenyl)-6-chloro-2,4-dioxo-1H-pyrrolo[3,2-d]pyrimidin-3(2H,4H,5H)-yl)benzoic acid;
5-(4-(2-aminothiazol-4-yl)phenyl)-6-chloro-3-(2-fluorophenyl)-1H-pyrrolo[3,2-d]pyrimidine-2,4(3H,5H)-dione;
5-(4-(2-aminothiazol-4-yl)phenyl)-6-chloro-3-(4-methoxy-2-methylphenyl)-1H-pyrrolo[3,2-d]pyrimidine-2,4(3H,5H)-dione;
5-(4-(2-aminothiazol-4-yl)phenyl)-6-chloro-3-(4-methoxyphenyl)-1H-pyrrolo[3,2-d]pyrimidine-2,4(3H,5H)-dione;
5-(4-(2-aminothiazol-4-yl)phenyl)-6-chloro-3-(2,5-dimethoxyphenyl)-1H-pyrrolo[3,2-d]pyrimidine-2,4(3H,5H)-dione;
5-(4-(2-aminothiazol-4-yl)phenyl)-6-chloro-3-(3-(methylthio)phenyl)-1H-pyrrolo[3,2-d]pyrimidine-2,4(3H,5H)-dione; and
5-(4-(2-aminothiazol-4-yl)phenyl)-6-chloro-3-(m-tolyl)-1H-pyrrolo[3,2-d]pyrimidine-2,4(3H,5H)-dione; and
5-(4-(6-aminopyridin-2-yl)phenyl)-6-chloro-3-(3-methoxy-2-methylphenyl)-1H-pyrrolo[3,2-d]pyrimidine-2,4(3H,5H)-dione; and
5-(4-(2-aminothiazol-4-yl)phenyl)-6-chloro-3-(3-hydroxyphenyl)-1H-pyrrolo[3,2-d]pyrimidine-2,4(3H,5H)-dione.
   or a salt thereof.

Compounds of the invention have been found to activate AMPK and may therefore be useful in the treatment of diabetes, metabolic syndrome, atherosclerosis, dyslipidaemia, obesity, hypertension, cerebral ischemia, cognitive defect and cancer.

Within the context of the present invention, the terms describing the indications used herein are classified in the Merck Manual of Diagnosis and Therapy, 17^{th} Edition and/or the International Classification of Diseases 10^{th} Edition (ICD-10). The various subtypes of the disorders mentioned herein are contemplated as part of the present invention.

In one aspect, the invention provides a compound of formula (I) or a pharmaceutically acceptable salt thereof for use in medical therapy.

In one aspect, the invention provides the use of a compound of formula (I) or a pharmaceutically acceptable salt thereof in the manufacture of a medicament for the treatment or prophylaxis of a disease or a condition mediated by AMPK activation.

In another aspect, the invention provides the use of a compound of formula (I) or a pharmaceutically acceptable salt thereof in the manufacture of a medicament for the treatment or prophylaxis of diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof in the manufacture of a medicament for the treatment or prophylaxis of Type II diabetes, dyslipidaemia and cancer.

In one aspect, the invention provides a compound of formula (I) or a pharmaceutically acceptable salt thereof for use in a method of treatment or prophylaxis of a disease or a condition mediated by AMPK activation.

In another aspect, the invention provides a compound of formula (I) or a pharmaceutically acceptable salt thereof for use in a method of treatment or prophylaxis of diabetes, metabolic syndrome, atherosclerosis, dyslipidaemia, obesity, hypertension, cerebral ischemia, cognitive defect and cancer.

In another aspect, the invention provides a compound of formula (I) or a pharmaceutically acceptable salt thereof for use in a method of treatment or prophylaxis of Type II diabetes, dyslipidaemia and cancer.

In one aspect, the invention provides a method for the treatment of a disease or a condition susceptible to amelioration by an AMPK activator in a subject in need thereof comprising administering to said subject a therapeutically effective amount of a compound of formula (I) or pharmaceutically acceptable salt thereof.

In another aspect, the invention provides a method for the treatment of diabetes, metabolic syndrome, atherosclerosis, dyslipidaemia, obesity, hypertension, cerebral ischemia, cognitive defect and cancer in a subject in need thereof comprising administering to said subject a therapeutically effective amount of a compound of formula (I) or pharmaceutically acceptable salt thereof.

In another aspect, the invention provides a method for the treatment of Type II diabetes, dyslipidaemia and cancer in a subject in need thereof comprising administering to said subject a therapeutically effective amount of a compound of formula (I) or pharmaceutically acceptable salt thereof.

It will be appreciated that reference to “treatment” and “therapy” includes acute treatment.

In the context of the present invention the term “prophylaxis” refers to the alleviation of established symptoms and/or retardation of progression of the disease, and may include the suppression of symptom recurrence in an asymptomatic patient.

### Pharmaceutical Compositions

While it is possible that, for use in the methods of the invention, a compound of formula (I) or a pharmaceutically acceptable salt thereof may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

Accordingly, the present invention also includes a pharmaceutical composition comprising a) a compound of formula (I) or a pharmaceutically acceptable salt thereof and b) one or more pharmaceutically acceptable carriers.

The term “pharmaceutically acceptable carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers or diluents are well known in the pharmaceutical art, and are described, for example, in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, or in addition to, the carrier any suitable binder(s), lubricant(s), suspending agent(s) and/or coating agent(s).

The diluents, excipient and/or vehicle must be “pharmaceutically acceptable” in the sense of being compatible with the other ingredients of the composition and not deleterious to the recipient thereof.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use.

Examples of pharmaceutically acceptable diluent(s) useful in the compositions of the invention include, but are not limited to water, ethanol, propylene glycol and glycerine.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of pharmaceutically acceptable suspending agents useful in the compositions of the invention include, but are not limited to, sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non aqueous vehicles (which may include edible oils), for example almond oil, oily esters such as glycerine, propylene glycol, or ethyl alcohol; preservatives, for example methyl or propyl p-hydroxybenzoate or sorbic acid.

Examples of pharmaceutically acceptable coating materials useful in the compositions of the invention include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof.

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

The present invention relates to a pharmaceutical composition for use in a method of treatment of Type II diabetes, dyslipidaemia or cancer comprising a compound of formula (I) or a pharmaceutically acceptable salt thereof.

The present invention further relates to a pharmaceutical composition comprising a) 10 to 2000 mg of a compound of formula (I) or a pharmaceutically acceptable salt thereof, and b) 0.1 to 2 g of one or more pharmaceutically acceptable carriers.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any suitable route, and include those in a form adapted for oral, parenteral, transdermal, inhalation, sublingual, topical, implant, nasal, enterally (or other mucosally) administration to mammals including humans. The pharmaceutical compositions may be formulated in conventional manner using one or more pharmaceutically acceptable carriers or excipients. In one aspect, the pharmaceutical composition is formulated for oral administration.

The compositions may be in the form of tablets, capsules, powders, granules, lozenges, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

The compounds of the invention may also, for example, be formulated as suppositories containing conventional suppository bases e.g. cocoa butter or other glyceride for use in human or veterinary medicine or as pessaries e.g., containing conventional pessary bases.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant e.g. sorbitan trioleate.

Capsules and cartridges (made, for example, from gelatin) for use in an inhaler or insufflator may be formulated to contain a powder mix of the compound and a suitable powder base such as lactose or starch.

Advantageously, agents such as a local anaesthetic, preservative and buffering agent can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compounds of the invention may be administered for immediate-, delayed-, modified-, sustained-, pulsed- or controlled-release applications.

The compositions may contain from 0.1% by weight, preferably from 10-60% by weight, of the active ingredient, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

The compounds of formula (I) or pharmaceutically acceptable salt(s) thereof may also be used in combination with other therapeutic agents. The invention thus provides, in a further aspect, a combination comprising a) a compound of formula (I) or pharmaceutically acceptable salt thereof and b) one or more further therapeutically active agent(s).

The combinations referred to above may conveniently be presented for use in the form of a pharmaceutical composition and thus pharmaceutical compositions comprising a combination as defined above together with one or more pharmaceutically acceptable carriers thereof represent a further aspect of the invention.

Compounds of the invention may be administered in combination with other therapeutically active agents. Preferred therapeutic agents are selected from the list: bisguanidine, metformin, a DPP-IV inhibitor, sitagliptin, an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA: cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

When the compound of formula (I) or pharmaceutically acceptable salt thereof is used in combination with a second therapeutically active agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention.

The individual components of such combinations may be administered either sequentially or simultaneously in separate or combined pharmaceutical formulations by any convenient route.

When administration is sequential, either the AMPK activator or the second therapeutically active agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

### Methods of Preparation

Compounds of formula (I) and salts thereof may be prepared by the general methods outlined hereinafter or any method known in the art, said methods constituting a further aspect of the invention. R¹ to R⁴ and X are as defined above unless otherwise specified. Throughout the specification, general formulae are designated by Roman numerals (I), (II), (III), (IV) etc.

In a general process, compounds of formula (I) or salts thereof, may be prepared according to reaction scheme 1 by reacting compounds of formula (II) or salts thereof, where P₁ is a suitable protecting group such as methyl, in the presence of an inorganic acid or base such as HCl or KOH in a suitable solvent such as ethanol or methanol (suitably at reflux).

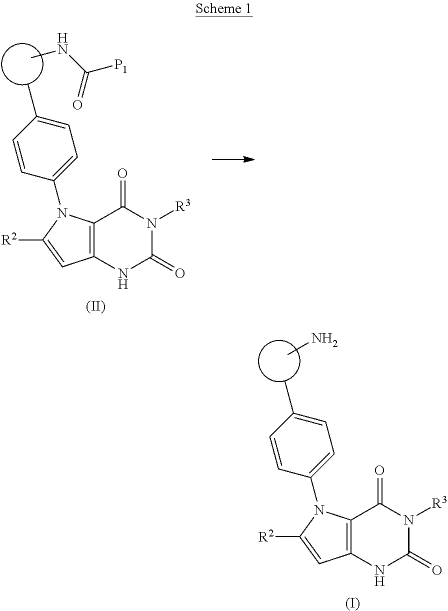

Compounds of formula (II) or salts thereof, may be prepared according to reaction scheme 2 by reacting compounds of formula (III) or salts thereof (where OL is a suitable leaving group such as ethoxy and C(O)P₁ is a suitable protecting group such as acetyl), in the presence of an inorganic base such as NaOMe or NaOEt in a suitable solvent such as ethanol or methanol (suitably at 60 to 90° C.).

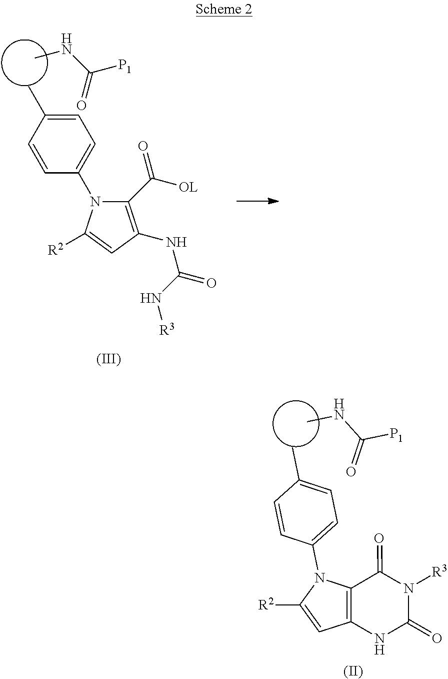

Compounds of formula (I), may be alternatively prepared according to reaction scheme 3 by reacting compounds of formula (III) or salts thereof (where OL is a suitable leaving group such as ethoxy and C(O)P₁ is a suitable protecting group such as acetyl), in the presence of an inorganic base such as NaOMe or NaOEt in a suitable solvent such as ethanol or methanol (suitably at 80 to 90° C.) then in the presence of an inorganic acid or base such as HCl or KOH in a suitable solvent such as ethanol or methanol (suitably at reflux).

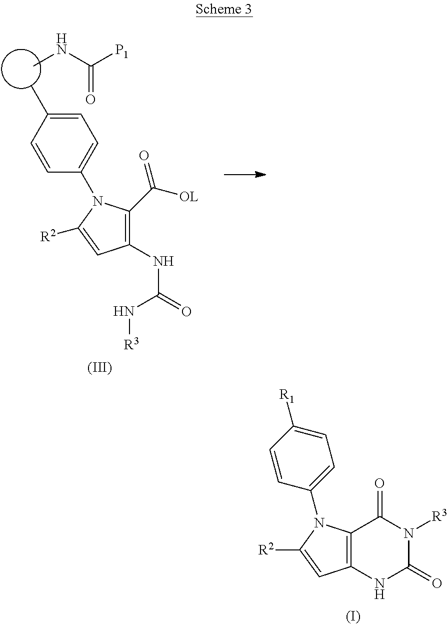

Compounds of formula (III) or salts thereof (wherein OL is a suitable leaving group such as ethoxy and C(O)P₁ is a suitable protecting group such as acetyl), may be prepared according to reaction scheme 4 by reacting compounds of formula (IV) or salts thereof (wherein L is a suitable leaving group and P₁ is a suitable protecting group), with the appropriate commercially available isocyanate (V) in a suitable solvent such as xylene or toluene (suitably at 80 to 160° C.).

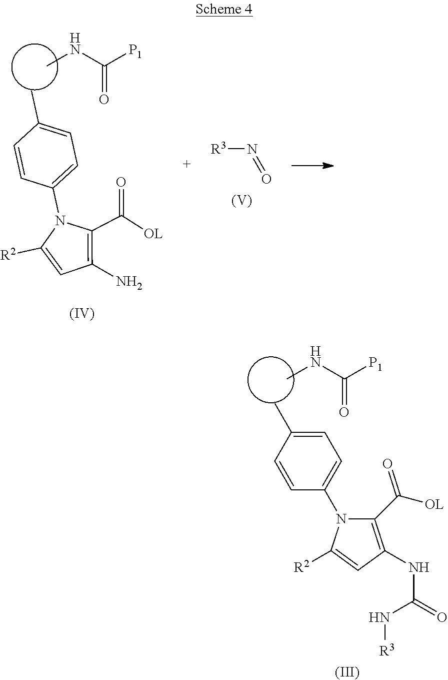

Compounds of formula (V) or salts thereof are commercially available or may be prepared by methods known in the literature or processes known to those skilled in the art.

Compounds of formula (III) or salts thereof (wherein OL is a suitable leaving group such as ethoxy and C(O)P₁ is a suitable protecting group such as acetyl), may be alternatively prepared in two steps according to reaction scheme 5 by:
(i) reacting compounds of formula (IV) or salts thereof where OL is a suitable leaving group and C(O)P₁ is a suitable protecting group) with 4-nitrophenyl carbonochloridate in a suitable solvent such as DCM in order to obtain intermediate (VII); then
(ii) reacting intermediate (VII) with the appropriate amine (VI) in a suitable solvent such as THF in a microwave reactor at high temperature.

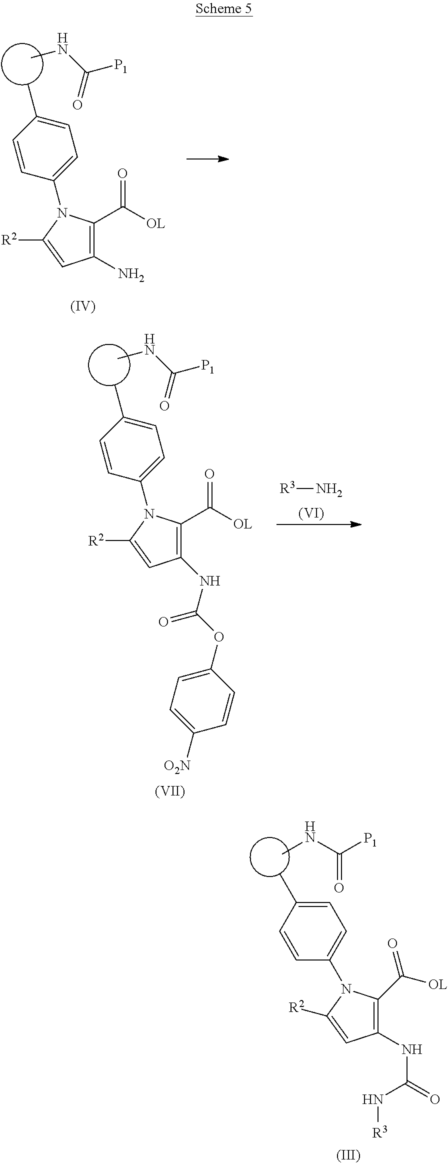

Compounds of formula (VI) are commercially available or may be prepared by methods known in the literature or processes known to those skilled in the art.

Compounds of formula (IV) or salts thereof (wherein OL is a suitable leaving group such as ethoxy) may be prepared according to reaction scheme 6 by reacting compounds of formula (VIII) with the appropriate bromo derivative (IX) (wherein C(O)P₁ is a suitable protecting group such as acetyl) in the presence of an inorganic base such as cesium carbonate or sodium carbonate and a catalyst (such as Pd(Ph₃P)₄) in a suitable solvent such as 1,4-dioxane at reflux.

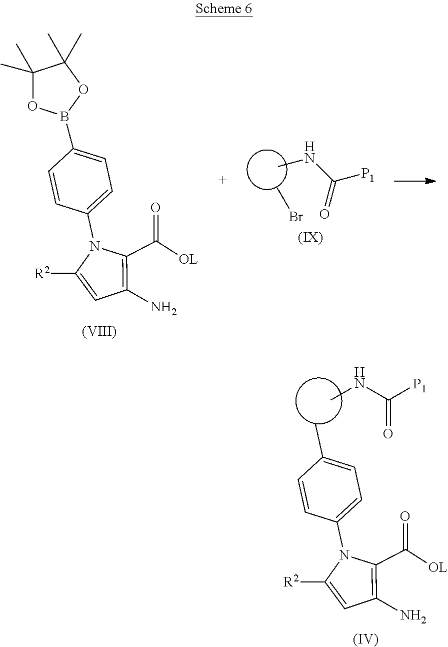

Compounds of formula (IX) are commercially available or may be prepared by methods known in the literature or processes known to those skilled in the art.

Alternatively, compounds of formula (IV) or salts thereof (wherein OL is a suitable leaving group such as ethoxy) may be prepared according to reaction scheme 6a by first reacting compounds of formula (XII) (wherein L is a suitable leaving group such as ethyl and P₂ is a suitable protecting group such as tertbutylcarbamate) with boronic acid derivative (XV) in the presence of a copper catalyst such as copper acetate and a base such as pyridine or triethylamine in a suitable solvent such as DCM (suitably at room temperature) to form the protected derivative of formula (XVIII). Compounds of formula (IV) or salts thereof (wherein OL is a suitable leaving group such as ethoxy and C(O)P₁ is a suitable protecting group such as acetyl) may be obtained treating compounds of formula (XVIII) with an acid such as TFA in a suitable solvent such as DCM (suitably at room temperature).

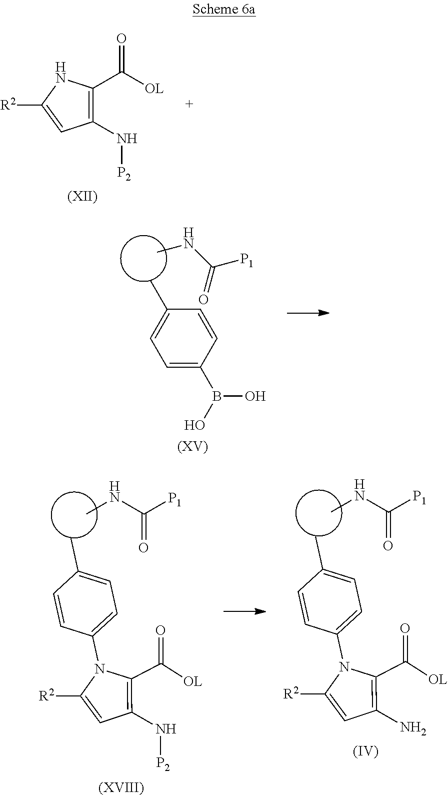

Compounds of formula (VIII) may be prepared according Scheme 7 by reacting compounds of formula (X), or salts thereof (wherein OL is a suitable leaving group such as ethoxy) with 4,4,4′,4′,5,5,5′,5′-octamethyl-2,2′-bi(1,3,2-dioxaborolane) in the presence of a catalyst such as Pd(dppf)Cl₂ and an inorganic base such as potassium acetate in a suitable solvent such as 1,4-dioxane at 85-100° C.

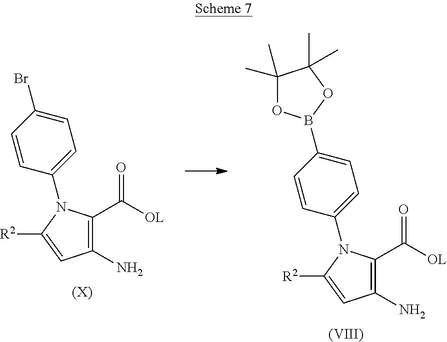

Compounds of formula (X) or salts thereof (wherein OL is a suitable leaving group such as ethoxy) may be prepared according to reaction scheme 8 by reacting compounds of formula (XI) or salts thereof (wherein OL is a suitable leaving group such as ethoxy and P₂ is a suitable protecting group such as acetyl) in the presence of an acid such as HCl in a suitable solvent such as ethanol (suitably at reflux).

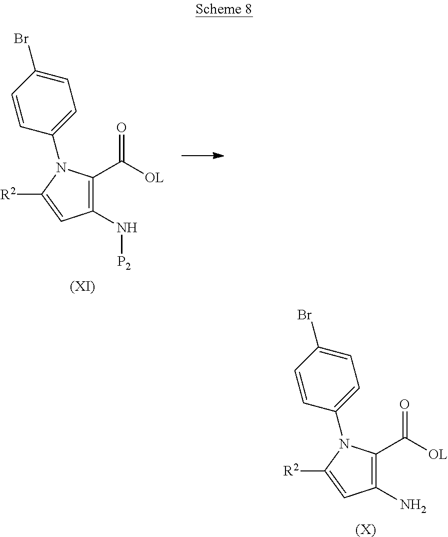

Compounds of formula (XI) may be prepared according to reaction scheme 9 by reacting compounds of formula (XII) (wherein OL is a suitable leaving group such as ethoxy and P₂ is a suitable protecting group such as acetyl) with boronic acid derivatives (XIII) in the presence of a copper catalyst such as copper acetate and a base such as pyridine or triethylamine in a suitable solvent such as DCM (suitably at room temperature).

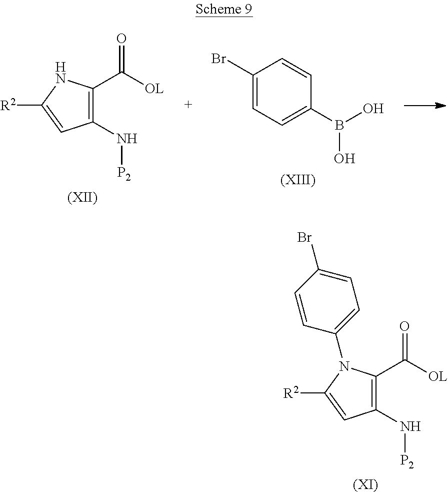

Compounds of formula (XIII) are commercially available or may be prepared by methods known in the literature or processes known to those skilled in the art.

Compounds of formula (XII), wherein R² is chloro (formula (XIIa), may be prepared according to reaction scheme 10 by reacting a compound of formula (XIV), (wherein OL is a suitable leaving group such as ethoxy and P₂ is a suitable protecting group such as acetyl), with N-chlorosuccinimide (NCS) in a suitable solvent such as chloroform or THF (suitably at room temperature).

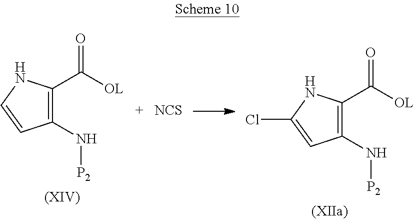

Compounds of formula (XII) wherein R² is —CN (formula (XIIb) can be prepared from compounds of formula (XIIa) according the scheme 11 by treatment with zinc cyanide and a catalyst (such as palladium trifluoroacetate and 2-(di-t-butylphosphino)-1,1′-binaphtyl as a ligand) in a suitable solvent such as N,N-dimethylacetamide.

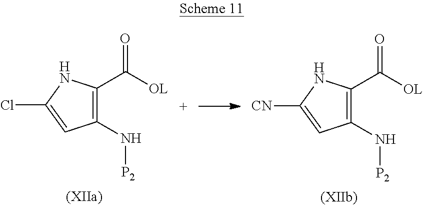

Compounds of formula (XII) wherein R² is H or C₁₋₄alkyl or halo (other than chloro) are commercially available or may be prepared by methods known in the literature or processes known to those skilled in the art.

Compounds of formula (XIV) and NCS are commercially available or may be prepared by methods known in the literature or processes known to those skilled in the art.

Compounds of formula (XV) may be prepared in two steps according to reaction scheme 12 by reacting compounds of formula (XVI) with 4,4,4′,4′,5,5,5′,5′-octamethyl-2,2′-bi-1,3,2-dioxaborolane in the presence of an inorganic base such as potassium acetate and a catalyst (such as PdCl₂dppf.DCM) in a suitable solvent such as 1,4-dioxane (suitably at 100° C.) to form the 4,4,5,5-tetramethyl-2-(1,3,2-dioxaborolane) derivative of formula (XVII). Compounds of formula (XV) may be prepared by reacting the 4,4,5,5-tetramethyl-2-(1,3,2-dioxaborolane) derivative of formula (XVII) in the presence of sodium periodate and ammonium acetate in a suitable solvent such as an acetone/water mixture (suitably at RT).

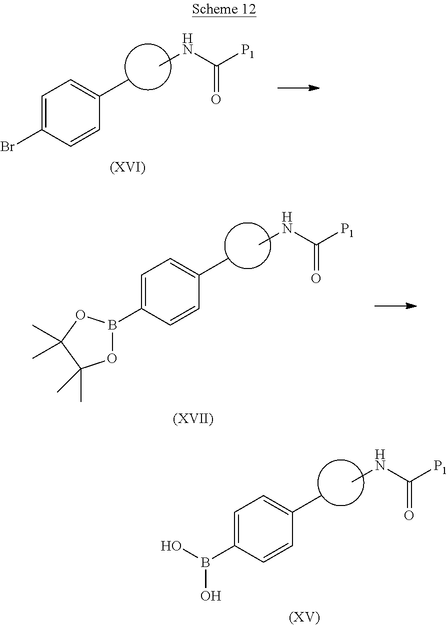

Compounds of formula (XVI) are commercially available compounds or can be made by methods known in the art (for instance J. Am. Chem. Soc. 1950, 72, 3722).

Compounds of formula (I), wherein R³ is 3-hydroxyphenyl (formula (Ia), may be prepared according to reaction scheme 13 by reacting a compound of formula (Ib) (a compound of formula (I) wherein R³ is 3-methoxyphenyl) with BBr₃ in a suitable solvent such as DCM (suitably at room temperature).

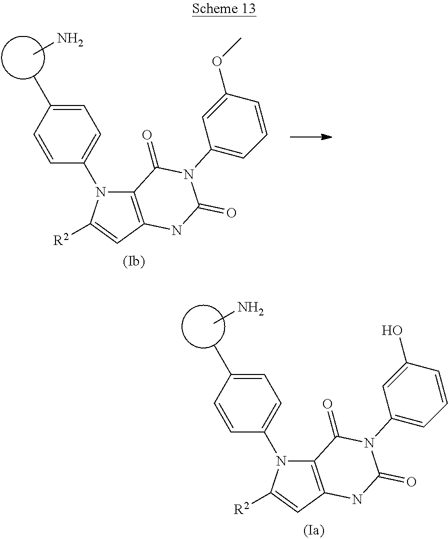

Further details for the preparation of compounds of formula (I) are found in the Examples section herein after.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Those skilled in the art will appreciate that in the preparation of compounds of formula (I) and/or solvates thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Various intermediate compounds used in the above-mentioned process, including but not limited to certain compounds of formulae (II), (XIV) and (XVI) constitute a further aspect of the present invention.

### DEFINITIONS

AcOH acetic acid
BBr₃ boron tribromide
CH₃CN acetonitrile
CDCl₃ deuterated chloroform
DCM dichloromethane
DMAP dimethylaminopyridine

### DMF N,N-dimethylformamide

DMSO d6 deuterated dimethylsulfoxide
DMSO dimethylsulfoxide
Et₃N triethylamine
Et₂O diethyl ether
EtOAc/AcOEt ethyl acetate
EtOH ethanol
EtONa sodium ethoxide
h hours
H₂SO₄ sulphuric acid
H₂O water
HATU O-(7-azabenzotriazol-1-yl)-1,1,3,3-tetramethyluronium hexafluorophosphate
HCl hydrochloric acid
HPLC high performance liquid chromatography
HRMS high resolution mass spectroscopy
iPr₂O di-isopropyl ether
LCMS liquid chromatography mass spectroscopy
MeCN acetonitrile
MeOH methanol
MeONa sodium methoxide
NaHCO₃ sodium hydrogen carbonate
NaOH sodium hydroxide
Na₂SO₄ sodium sulphate

### NCS N-chlorosuccinimide

NMR Nuclear magnetic resonance
Pd(dppf)Cl_{2 [}1,1′-Bis(diphenylphosphino)ferrocene]dichloropalladium(II)
Pd(Ph₃P)₄ tetrakis(triphenylphosphine)palladium
RT room temperature
Rt retention time
Sat. saturated
SM starting material
TFA trifluoroacetic acid
THF tetrahydrofuran
TLC thin-layer chromatography
TMS tetramethylsilane

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Regardless of how the preparation of compounds are represented in the present specification no inference can be drawn that particular batches (or mixtures of two or more batches) of intermediates were used in the next stage of the preparation. The examples and intermediates are intended to illustrate the synthetic routes suitable for preparation of the same, to assist the skilled persons understanding of the present invention.

Where reference is made to the use of a “similar” procedure, as will be appreciated by those skilled in the art, such a procedure may involve minor variation, for example reaction temperature, reagent/solvent amount, reaction time, work-up conditions or chromatographic purification conditions.

Compounds are named using ACD/Name PRO 6.02 chemical naming software (Advanced Chemistry Development Inc., Toronto, Ontario, M5H2L3, Canada).

### Analytical Method LC-MS

(a) Analytical HPLC was conducted on a X-terra MS C18 column (2.5 μm 3×30 mm id) eluting with 0.01M ammonium acetate in water (solvent A) and 100% acetonitrile (solvent B) using the following elution gradient: 0 to 4 minutes, 5% B to 100% B; 4 to 5 minutes, 100% B at a flow rate of 1.1 mL/min with a temperature of 40° C. The mass spectra (MS) were recorded on a Micromass ZQ-LC mass spectrometer using electrospray positive ionisation [ES+ve to give MH⁺ molecular ion] or electrospray negative ionisation [ES−ve to give (M−H)⁻ molecular ion] modes.

### Or

(b) Analytical HPLC was conducted on a X-Terra MS C18 column (3.5 μm 30×4.6 mm id) eluting with 0.01M ammonium acetate in water (solvent A) and 100% methanol (solvent B), using the following elution gradient 0 to 7.5 minutes 10 to 100% B, 7.5-10 minutes 100% B, 10.5-12 min 10% B at a flow rate of 1.4 ml/minute.

The mass spectra (MS) were recorded on a Waters ZQ mass spectrometer using electrospray positive ionisation [ES+ to give MH+ molecular ions] or electrospray negative ionisation [ES- to give (M−H)-molecular ions] modes.

### Analytical LC-HRMS

### Methods:

(a) Analytical HPLC was conducted on a LUNA 3u C18 column (2.5 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5% B to 100% B; 3.5 to 4 minutes, 100% B; 4 to 4.5 minutes, 100% B to 5% B; 4.5 to 5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES−ve to give (M−H)⁻ molecular ion] modes.

### Or

(b) Analytical HPLC was conducted on a X-Bridge C18 column (2.5 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5% B to 100% B; 3.5 to 4 minutes, 100% B; 4 to 4.5 minutes, 100% B to 5% B; 4.5 to 5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES−ve to give (M−H)⁻ molecular ion] modes.

¹H NMR spectra were acquired on a 300 MHz or a 400 MHz Bruker spectrometer. Sample was dissolved in DMSO-d6 or CDCl₃ and chemical shifts (δ) were reported in ppm relative to the TMS signal at δ=0 ppm. Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

The following non-limiting Examples illustrate the present invention.

### Intermediate 1

### Ethyl 3-(acetylamino)-1H-pyrrole-2-carboxylate

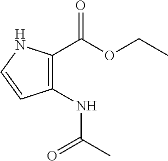

Method A: To a suspension of ethyl 3-amino-1H-pyrrole-2-carboxylate hydrochloride (1 g, 4.98 mmol, commercially available from Combi-Blocks) in DCM (50 mL) at 0° C. was added drop-wise triethylamine (2 mL, 14.43 mmol) and acetyl chloride (0.45 mL, 6.31 mmol). The reaction mixture was then stirred from 0° C. to RT for 12 hours before being quenched with 1N HCl. The organic layer was separated and washed successively with sat. NaHCO₃ and brine, dried over Na₂SO₄, filtered and concentrated under reduced pressure. The product was purified by chromatography. The sample was loaded on 100 g silica column and then the purification was carried out using DCM/MeOH:100/0 to 90/10. The appropriate fractions were combined and evaporated in vacuo to give the required product ethyl 3-(acetylamino)-1H-pyrrole-2-carboxylate (0.99 g, 5.05 mmol, 100% yield) as a yellow solid. ¹H NMR: (CDCl₃, 400 Hz) δ 9.12 (br s, 1H), 8.78 (br s, 1H), 7.05 (s, 1H), 6.81 (s, 1H), 4.34 (q, J=7.0 Hz, 2H), 2.18 (s, 3H), 1.37 (t, J=7.0 Hz, 3H). LCMS: (M+H)⁺: 197; Rt: 1.93 min.

Method B: To a suspension of ethyl 3-amino-1H-pyrrole-2-carboxylate (commercially available from Combi-Blocks, 25 g, 131 mmol) in (DCM) (150 mL) at 0° C. was added triethylamine (40.1 mL, 289 mmol). After stirring for 10 minutes, a solution of acetyl chloride (10.26 mL, 144 mmol) in (DCM) (50 mL) was added dropwise. The reaction mixture was then stirred from 0° C. to RT for 3 hours before being quenched with sat NaHCO₃. More DCM was added to solubilise a precipitate. The organic layer was separated and washed with brine, dried over Na₂SO₄, filtered and concentrated under reduced pressure. The product was purified by recrystallisation in diisopropyl oxide to give ethyl 3-(acetylamino)-1H-pyrrole-2-carboxylate (22 g, 107 mmol, 81% yield) as a white powder. LCMS: (M+H)⁺: 197; Rt: 1.92 min

### Intermediate 2

### Ethyl 3-(acetylamino)-5-chloro-1H-pyrrole-2-carboxylate

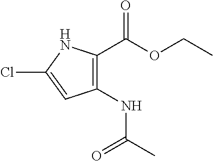

Method A: Ethyl 3-(acetylamino)-1H-pyrrole-2-carboxylate (Intermediate 1) (40 g, 204 mmol) was dissolved in chloroform (250 mL) and N-chlorosuccinimide (NCS) (28.6 g, 214 mmol) was added portion-wise. The mixture was stirred at RT for 1 h, and after TLC, was warmed to 35° C. during 2 hours. The mixture was then poured into water and extracted with DCM, dried over Na₂SO₄, and concentrated in vacuo. The mixture was triturated in DCM and the precipitate was filtered, washed with a small of DCM and washed with Et₂O to give ethyl 3-(acetylamino)-5-chloro-1H-pyrrole-2-carboxylate (20 g, 42% yield) as a white solid. ¹H NMR: (DMSO-d6, 300 Hz) δ 12.45 (br s, 1H), 9.23 (s, 1H), 6.68 (s, 1H), 4.27 (q, J=7.1 Hz, 2H), 2.08 (s, 3H), 1.30 (t, J=7.1 Hz, 3H). LCMS: (M+H)⁺: 231; Rt: 2.32 min.

Method B: To a solution of ethyl 3-(acetylamino)-1H-pyrrole-2-carboxylate (Intermediate 1) (10 g, 51.0 mmol) in chloroform (150 mL) was added slowly N-chlorosuccinimide (NCS) (7.49 g, 56.1 mmol) and the reaction mixture was stirred at RT for 48 hours. Water was added and the product was extracted with DCM. The organic layer was dried over Na₂SO₄, filtered and evaporated off. The residue was purified by chromatography on silica gel eluting with DCM/MeOH 100/0 to 90/10 to give the product ethyl 3-(acetylamino)-5-chloro-1H-pyrrole-2-carboxylate (5.8 g, 25.1 mmol, 49.3% yield) as white powder. LCMS: (M+H)⁺: 231; Rt: 2.30 min.

### Intermediate 3

### Ethyl 3-(acetylamino)-1-(4-bromophenyl)-5-chloro-1H-pyrrole-2-carboxylate

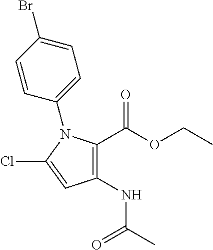 [SMILES: CCOC(=O)c1c(NC(C)=O)cc(Cl)n1-c1ccc(Br)cc1]

Method A: To a suspension of ethyl 3-(acetylamino)-5-chloro-1H-pyrrole-2-carboxylate (Intermediate 2) (200 mg, 0.867 mmol) and molecular sieves 4A (500 mg, 0.867 mmol) in DCM (5 mL) was added (4-bromophenyl)boronic acid (192 mg, 0.954 mmol), copper(II) acetate (173 mg, 0.954 mmol) and Et₃N (0.181 mL, 1.301 mmol). The reaction mixture was stirred at room temperature overnight. TLC and LCMS showed the reaction was incomplete. More (4-bromophenyl)boronic acid (192 mg, 0.954 mmol) was added every 2 hours until the reaction was complete (4 equiv. in total). The mixture was filtered on silica pad (DCM and MeOH) and the brown filtrate was concentrated. The residue was purified by chromatography on silica gel (interchim 12g) (DCM/MeOH 100/0 to 99/1) to give the product ethyl 3-(acetylamino)-1-(4-bromophenyl)-5-chloro-1H-pyrrole-2-carboxylate (350 mg, 0.862 mmol, 99% yield) as a light yellow oil. ¹H NMR: (DMSO-d6, 300 Hz) δ 9.50 (s, 1H), 7.70 (m, 2H), 7.30 (m, 2H), 7.01 (s, 1H), 3.98 (q, J=7.1 Hz, 2H), 2.13 (s, 3H), 0.91 (t, J=7.1 Hz, 3H). LCMS: (M+H)⁺: 385, 387; Rt: 3.83 min.

Method B: To a suspension of ethyl 3-(acetylamino)-5-chloro-1H-pyrrole-2-carboxylate (Intermediate 2) (5.8 g, 25.1 mmol) in DCM (250 mL) was added (4-bromophenyl)boronic acid (5.56 g, 27.7 mmol), copper(II) acetate (5.0 g, 27.7 mmol) and pyridine (3.05 mL, 37.7 mmol). The reaction mixture was stirred at room temperature for two days. (4-Bromophenyl)boronic acid (5.6 g, 27.7 mmol), copper(II) acetate (5.0 g, 27.7 mmol) and pyridine (3.05 mL, 37.7 mmol) were added again every 2 days (4.4 equiv. in total). Water was added and the product was extracted with DCM. The organic layer was dried over Na₂SO₄ filtered and evaporated off. The residue was purified by chromatography on silica gel eluting with DCM/MeOH 100/0 to 90/10 to give the product ethyl 3-(acetylamino)-1-(4-bromophenyl)-5-chloro-1H-pyrrole-2-carboxylate (2.8 g, 6.90 mmol, 27.4% yield) as a yellow oil. LCMS: (M+H)⁺: 387; Rt: 3.48 min.

### Intermediate 4

### Ethyl 3-amino-1-(4-bromophenyl)-5-chloro-1H-pyrrole-2-carboxylate

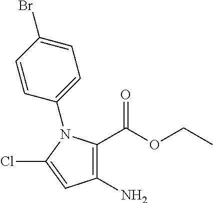 [SMILES: CCOC(=O)c1c(N)cc(Cl)n1-c1ccc(Br)cc1]

Method A: To a solution of ethyl 3-(acetylamino)-1-(4-bromophenyl)-5-chloro-1H-pyrrole-2-carboxylate (Intermediate 3) (9 g, 23.34 mmol) in ethanol (200 mL) was added concentrated HCl (9.58 mL, 117 mmol). The mixture was refluxed for 2 hours before being concentrated in vacuo and the precipitate was triturated in diethyl ether and filtered to give ethyl 3-amino-1-(4-bromophenyl)-5-chloro-1H-pyrrole-2-carboxylate (8.53 g, 96% yield) as a white solid. ¹H NMR: (DMSO-d6, 300 Hz) δ 7.67 (d, J=8.6 Hz, 2H), 7.24 (d, J=8.6 Hz, 2H), 6.17 (s, 1H), 3.97 (q, J=7.1 Hz, 2H), 0.98 (t, J=7.1 Hz, 3H). LCMS: (M+H)⁺: 343, 345; Rt: 3.74 min.

Method B: To a solution of ethyl 3-(acetylamino)-1-(4-bromophenyl)-5-chloro-1H-pyrrole-2-carboxylate (Intermediate 3) (1.3 g, 3.37 mmol) in ethanol (20 mL) was added concentrated HCl (1.38 mL, 16.9 mmol). The mixture was refluxed for 2 hours before being concentrated in vacuo and the precipitate was triturated in diethyl ether and filtered to give ethyl 3-amino-1-(4-bromophenyl)-5-chloro-1H-pyrrole-2-carboxylate (820 mg, 64% yield) as a white solid. LCMS: (M+H)⁺: 343, 345; Rt: 3.42 min.

### Intermediate 5

### Ethyl 3-amino-5-chloro-1-(4-(4,4,5,5-tetramethyl-1,3,2-dioxaborolan-2-yl)phenyl)-1H-pyrrole-2-carboxylate

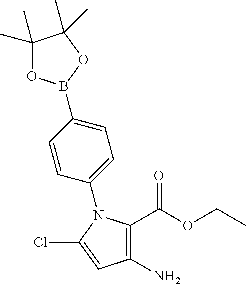 [SMILES: CCOC(=O)c1c(N)cc(Cl)n1-c1ccc(B2OC(C)(C)C(C)(C)O2)cc1]

To a solution of ethyl 3-amino-1-(4-bromophenyl)-5-chloro-1H-pyrrole-2-carboxylate (Intermediate 4) (38 g, 110.6 mmol) in 1,4-dioxane (400 mL) were added respectively 4,4,4′,4′,5,5,5′,5′-octamethyl-2,2′-bi(1,3,2-dioxaborolane) (42.1 g, 165.9 mmol), potassium acetate (32.5 g, 331.8 mmol), Pd(dppf)Cl₂ (8.1 g, 11.1 mmol). The reaction mixture was then heated to 85° C. for 16 h under nitrogen atmosphere before being filtered (washing with EtOAc) The combined organic layers were concentrated under reduced pressure. EtOAc and water were added and the aqueous layer was extracted with EtOAc (3×). The combined organic layers were dried, concentrated under reduced pressure and the resulting crude product was purified by column chromatography (pentane/ether:20/1) to give the title compound (21.22 g, 98% yield). LCMS: (M+H)⁺=391; Rt=2.38 min.

### Intermediate 6

### N-(6-Bromo-2-pyridinyl)acetamide

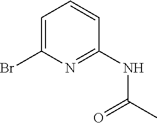

To a solution of 6-bromo-2-pyridinamine (5 g, 28.9 mmol) in DCM was added triethylamine (8.06 mL, 57.8 mmol) and acetyl chloride (3.08 mL, 43.3 mmol) was slowly added. The reaction mixture was stirred at room temperature overnight. Acetyl chloride (3.08 mL, 43.3 mmol) was added again and the reaction mixture was stirred for another 1 h. The organic layer was washed with water then solvent was removed under reduced pressure to give N-(6-bromo-2-pyridinyl)acetamide (6.44 g, 29.9 mmol, quantitative yield) as an orange solid. LCMS: (M+H)⁺=215, 217; Rt=2.15 min.

### Intermediate 7

### Ethyl 1-{4-[6-(acetylamino)-2-pyridinyl]phenyl}-3-amino-5-chloro-1H-pyrrole-2-carboxylate

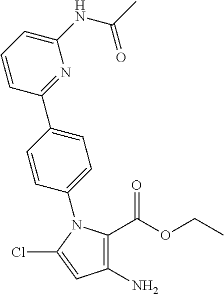 [SMILES: CCOC(=O)c1c(N)cc(Cl)n1-c1ccc(-c2cccc(NC(C)=O)n2)cc1]

Ethyl 3-amino-5-chloro-1-[4-(4,4,5,5-tetramethyl-1,3,2-dioxaborolan-2-yl)phenyl]-1H-pyrrole-2-carboxylate (Intermediate 5) (5 g, 12.80 mmol), N-(6-bromo-2-pyridinyl)acetamide (Intermediate 6) (3.58 g, 16.64 mmol), Na₂CO₃ (4.07 g, 38.4 mmol), Pd(Ph₃)₄ (0.739 g, 0.640 mmol) were mixed in 1,4-dioxane (20 mL) and water (10 mL). The reaction mixture was stirred at 100° C. overnight before being concentrated under reduced pressure. The residue was taken up in DCM and washed with water and brine. The organic layer was removed under reduced pressure. The resulting solid was then recrystallized in EtOH to give the title compound (3 g, 7.52 mmol, 58.8% yield) as a yellow solid. LCMS: (M+H)⁺=399, 401; Rt=3.25 min.

### Intermediate 8

### Ethyl 1-{4-[6-(acetylamino)-2-pyridinyl]phenyl}-5-chloro-3-[({[3-(methyloxy)phenyl]amino}carbonyl)amino]-1H-pyrrole-2-carboxylate

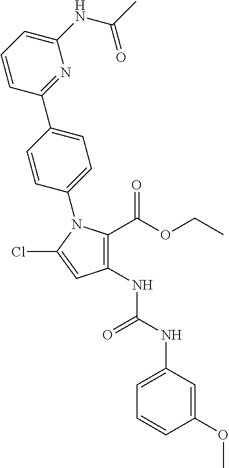 [SMILES: CCOC(=O)c1c(NC(=O)Nc2cccc(OC)c2)cc(Cl)n1-c1ccc(-c2cccc(NC(C)=O)n2)cc1]

To a solution of ethyl 1-{4-[6-(acetylamino)-2-pyridinyl]phenyl}-3-amino-5-chloro-1H-pyrrole-2-carboxylate (Intermediate 7) (350 mg, 0.88 mmol) in toluene (20 mL) was added 1-isocyanato-3-(methyloxy)benzene (157 mg, 1.05 mmol). The reaction mixture was stirred at 100° C. overnight before the resulting solid was filtered off. The filtrate was then concentrated under reduced pressure to give the title compound (321 mg, 0.586 mmol, 66.8% yield) as a cream solid. LCMS: (M+H)⁺=548; Rt=3.66 min.

### Intermediate 9

### Ethyl 3-((tert-butoxycarbonyl)amino)-1H-pyrrole-2-carboxylate

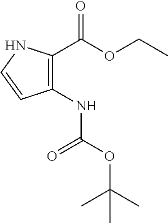 [SMILES: CCOC(=O)c1[nH]ccc1NC(=O)OC(C)(C)C]

To a solution of ethyl 3-amino-1H-pyrrole-2-carboxylate hydrochloride (10 g, 52.5 mmol, commercially available from Combi-Blocks) in dry pyridine (200 mL) at 0° C. was added successively DMAP (0.64 g, 5.2 mmol) and a solution of di-tert-butyl dicarbonate (22.9 g, 105 mmol) in pyridine (62 mL). The reaction mixture was stirred at RT overnight before being concentrated to dryness. The residue was taken up in EtOAc and washed successively with aqueous sat. NaHCO₃ and brine. The organic layer was dried over sodium sulfate, filtered and concentrated under reduced pressure. The crude product was purified by column chromatography (DCM 100% as eluant) to give the title compound (11.8 g, 89% yield) as a white solid. ¹H NMR (400 MHz, DMSO-d6) δ ppm 11.51 (br s, 1H), 8.32 (br s, 1H), 6.89 (s, 1H), 6.51 (br s, 1H), 4.25 (q, J=7.1 Hz, 2H), 1.46 (s, 9H), 1.29 (t, J=7.1 Hz, 3H).

### Intermediate 10

### Ethyl 3-((tert-butoxycarbonyl)amino)-5-chloro-1H-pyrrole-2-carboxylate

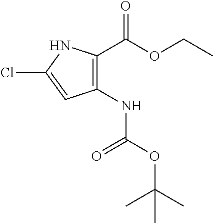 [SMILES: CCOC(=O)c1[nH]c(Cl)cc1NC(=O)OC(C)(C)C]

To a solution of ethyl 3-((tert-butoxycarbonyl)amino)-1H-pyrrole-2-carboxylate (Intermediate 9) (1 g, 3.9 mmol) in THF (20 mL) was added NCS (0.0.55 g, 4.13 mmol). The reaction mixture was stirred at 30° C. for 17 h before being diluted with EtOAc (100 mL). The organic layer was washed twice with brine, dried over sodium sulphate, filtered and concentrated under reduced pressure. The crude solid was purified by column chromatography (cyclohexane/EtOAc:9/1 as eluant) to give the desired product ethyl 3-((tert-butoxycarbonyl)amino)-5-chloro-1H-pyrrole-2-carboxylate (0.94 g, 84% yield). ¹H NMR (400 MHz, DMSO-d6) δ ppm 12.40 (br s, 1H), 8.35 (s, 1H), 6.48 (br s, 1H), 4.25 (q, J=7.1 Hz, 2H), 1.46 (s, 9H), 1.29 (t, J=7.1 Hz, 3H).

### Intermediate 11

### 4-(4-Bromophenyl)-1,3-thiazol-2-amine hydrobromide

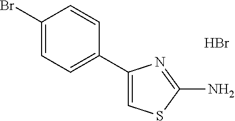 [SMILES: Nc1nc(-c2ccc(Br)cc2)cs1 ; Br]

To a suspension of 2-bromo-1-(4-bromophenyl)ethanone (50 g, 180 mmol) in ethanol (250 mL) was added thiourea (13.7 g, 180 mmol), the reaction was stirred 5 h at reflux. The reaction was cooling until precipitation (72 h). The precipitate was filtered, washed with ethanol and diethylether then dried to give the title compound as 5-(4-bromophenyl)-1,3-thiazol-2-amine hydrobromide (58.5 g, 174 mmol; 97% yield) as a white solid. ¹H NMR: (DMSO, 400 Hz) δ 7.7 (s, 4H), 7.3 (s, 1H).

### Intermediate 12

### N-[4-(4-bromophenyl)-1,3-thiazol-2-yl]acetamide

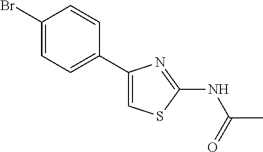 [SMILES: CC(=O)Nc1nc(-c2ccc(Br)cc2)cs1]

To a solution of 5-(4-bromophenyl)-1,3-thiazol-2-amine hydrobromide, (Intermediate 11) (57 g, 170 mmol) and pyridine (123 mL, 1.53 mol) in DMF (500 mL) was added dropwise at 0° C. acetic anhydride (22.4 mL, 237 mmol) in DMF (350 mL). The reaction was stirred at RT overnight. Acetic anhydride (8 mL, 85 mmol) in DMF (50 mL) was added dropwise and the reaction was stirred 24 h at RT. The reaction mixture was concentrated in vacuo and the solid residue was poured in water and saturated solution of NaHCO₃ was added. The precipitated was filtered, washed with water and dried to give the title compound N-[5-(4-bromophenyl)-1,3-thiazol-2-yl]acetamide (48.6 g, 163.5 mmol; 96% yield) as a white solid. ¹H NMR: (DMSO, 400 Hz) δ 7.8 (m, 2H), 7.6 (m, 3H); 2.2 (s, 3H).

### Intermediate 13

### N-{4-[4-(4,4,5,5-Tetramethyl-1,3,2-dioxaborolan-2-yl)phenyl]-1,3-thiazol-2-yl}acetamide

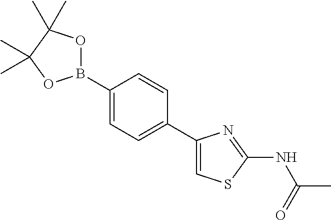 [SMILES: CC(=O)Nc1nc(-c2ccc(B3OC(C)(C)C(C)(C)O3)cc2)cs1]

A suspension of N-[5-(4-bromophenyl)-1,3-thiazol-2-yl]acetamide, (Intermediate 12), (20 g, 67 mmol), [1.1′-bis(diphenylphosphino)ferrocene]dichloropalladium(II), complex with dichloromethane (PdCl₂dppf.DCM) (2.75 g, 3.4 mmol), 4,4,4′,4′,5,5,5′,5′-octamethyl-2,2′-bi-1,3,2-dioxaborolane (21.4 g, 84 mmol) and potassium acetate (19.8 g, 202 mmol) in dioxane (673 mL) was purged 3 times with argon then stirred 17 h at 100° C. The reaction mixture was diluted with EtOAc, washed with brine and dried over Na₂SO₄, filtered and concentrated under reduced pressure. Purification by flash chromatography of 10 g of crude compound using chloroform/acetic acid 100% to 98/2 as gradient gave the title compound (6.8 g, 19.75 mmol, 68% purification yield). ¹H NMR: (CDCl₃, 400 Hz) δ 7.8 (d, 2H, J=7.6 Hz), 7.6 (d, 2H, J=7.6 Hz), 7.1 (s, 1H), 2.1 (s, 3H), 1.3 (s, 12H).

### Intermediate 14

### {4-[2-(Acetylamino)-1,3-thiazol-4-yl]phenyl}boronic acid

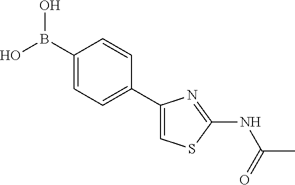 [SMILES: CC(=O)Nc1nc(-c2ccc(B(O)O)cc2)cs1]

To a solution of N-{5-[4-(4,4,5,5-tetramethyl-1,3,2-dioxaborolan-2-yl)phenyl]-1,3-thiazol-2-yl}acetamide, (Intermediate 13) (6.8 g, 19.8 mmol) in acetone (200 mL) was added water (12 mL), 1M solution of ammonium acetate (90 mL), and sodium periodate (NalO₄) (12.7 g, 53.3 mmol). The reaction was stirred 60 h at RT. The mixture was filtered and the solid residue was washed with acetone and water. The filtrate was concentrated to dryness, diluted with EtOAc, washed with brine and dried over MgSO₄, filtered and concentrated under reduced pressure to give the title compound (5.2 g, 19.8 mmol, 100% yield) as a cream solid. ¹H NMR: (DMSO, 400 Hz) δ 8.04 (m, 2H), 7.85 (m, 4H), 7.64 (s, 1H), 2.10 (s, 3H).

### Intermediate 15

### Ethyl 1-{4-[2-(acetylamino)-1,3-thiazol-4-yl]phenyl}-5-chloro-3-({[(1,1-dimethylethyl)oxy]carbonyl}amino)-1H-pyrrole-2-carboxylate

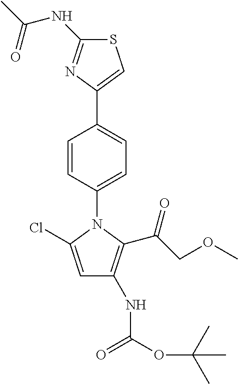 [SMILES: COCC(=O)c1c(NC(=O)OC(C)(C)C)cc(Cl)n1-c1ccc(-c2csc(NC(C)=O)n2)cc1]

To a solution of ethyl 3-((tert-butoxycarbonyl)amino)-5-chloro-1H-pyrrole-2-carboxylate, (Intermediate 10) (800 mg, 2.77 mmol) in DCM (80 mL), isopropanol (20 mL) was added {4-[2-(acetylamino)-1,3-thiazol-5-yl]phenyl}boronic acid, (Intermediate 14) (1.45 g, 5.55 mmol), copper(II) acetate (1 g, 5.55 mmol) and triethylamine (1.6 mL, 11 mmol). The mixture was stirred 48 h at RT with air bubbling. The reaction mixture was evaporated, diluted with ethyl acetate, washed with brine and dried over Na₂SO₄, filtered and concentrated under reduced pressure. Purification by flash chromatography using petroleum ether/EtOAC 4:1 as gradient gave the title compound (800 mg, 1.58 mmol, 57% yield). ¹H NMR: (CDCl₃, 400 Hz) δ 12.28 (brs, 2H), 8.75 (s, 1H), 8.00-7.34 (m, 4H), 6.96 (d, 1H), 3.94 (q, 2H), 2.17 (s, 3H), 1.49 (s, 9H), 0.82 (t, 3H).

### Intermediate 16

### Ethyl 1-{4-[2-(acetylamino)-1,3-thiazol-4-yl]phenyl}-3-amino-5-chloro-1H-pyrrole-2-carboxylate trifluoroacetate salt

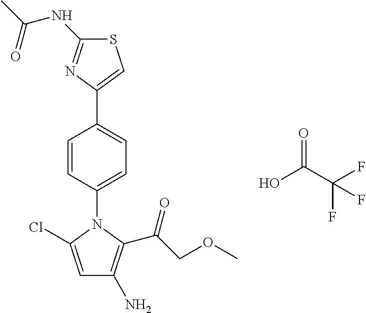 [SMILES: COCC(=O)c1c(N)cc(Cl)n1-c1ccc(-c2csc(NC(C)=O)n2)cc1 ; O=C(O)C(F)(F)F]

To a solution of ethyl 1-{4-[2-(acetylamino)-1,3-thiazol-5-yl]phenyl}-5-chloro-3-({[(1,1-dimethylethyl)oxy]carbonyl}amino)-1H-pyrrole-2-carboxylate, (Intermediate 15), (1.2 g, 2.37 mmol) in DCM (20 mL) was added TFA (4 mL, 35 mmol). The reaction was stirred overnight at RT. The reaction mixture was evaporated and purification by flash chromatography using petroleum ether/EtOAc 7:3 to 1:1 as gradient gave the title compound (650 mg, 1.25 mmol, 53% yield) as a light yellow solid. ¹H NMR: (DMSO, 400 Hz) δ 12.27 (s, 1H), 7.95 (d, 2H, J=8.6 Hz), 7.68 (s, 1H), 7.25 (d, 2H, J=8.6 Hz), 5.9 (s, 1H), 3.92 (q, 2H, J=7.07 Hz), 2.18 (s, 3H), 0.90 (t, 3H, J=7.07 Hz).

### Intermediate 17

### Ethyl 1-{4-[2-(acetylamino)-1,3-thiazol-4-yl]phenyl}-5-chloro-3-[({[3-(methyloxy)phenyl]amino}carbonyl)amino]-1H-pyrrole-2-carboxylate

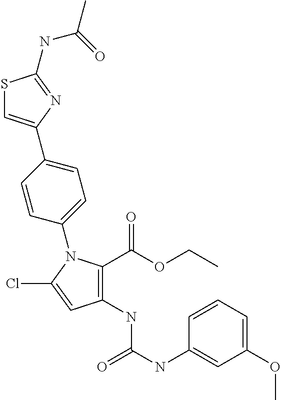 [SMILES: CCOC(=O)c1c(NC(=O)Nc2cccc(OC)c2)cc(Cl)n1-c1ccc(-c2csc(NC(C)=O)n2)cc1]

To a solution of ethyl 1-{4-[2-(acetylamino)-1,3-thiazol-4-yl]phenyl}-3-amino-5-chloro-1H-pyrrole-2-carboxylate trifluoroacetate (Intermediate 16) (7 g, 17.3 mmol) in toluene (20 mL) was added 1-isocyanato-3-(methyloxy)benzene (3.1 g, 20.7 mmol). The reaction mixture was stirred at 80° C. overnight before the resulting solid was filtered off. The filtrate was then concentrated under reduced pressure and the residue was triturated in DCM, filtered and dried to give the title compound (9.2 g, 16.61 mmol, 96% yield) as a yellow powder. LCMS: (M+H)⁺=554; Rt=3.59 min.

Intermediates 18 to 27 were prepared by methods analogous to that described for intermediate 17 from ethyl 1-{4-[2-(acetylamino)-1,3-thiazol-4-yl]phenyl}-3-amino-5-chloro-1H-pyrrole-2-carboxylate trifluoroacetate (Intermediate 16) and the appropriate isocyanate.

TABLE 1

| Intermediate | Formula | From | Physical Data |
|---|---|---|---|
| Intermediate 18 Ethyl 1-(4-(2- acetamidothiazol-4- yl)phenyl)-5-chloro-3- (3-(3- (methoxycarbonyl) phenyl)ureido)-1H- pyrrole-2-carboxylate | 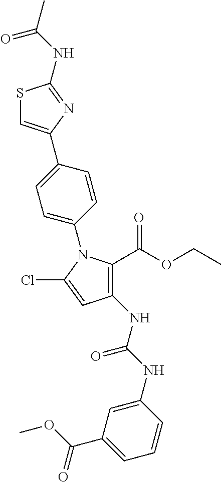 [SMILES: CCOC(=O)c1c(NC(=O)Nc2cccc(C(=O)OC)c2)cc(Cl)n1-c1ccc(-c2csc(NC(C)=O)n2)cc1] | Intermediate 16 and Methyl 3- isocyanato- benzoate | ¹H NMR (400 MHz, DMSO-d6) δ ppm 12.28 (br s, 1H), 10.14 (br s, 1H), 9.19 (br s, 1H), 8.22 (s, 2H), 8.00 (d, 2H), 7.73-7;37 (m, 5H), 7.07 (s, 1H), 3.97 (q, J = 7.1 Hz, 2H), 3.87 (s, 3H), 3.29 (s, 3H), 0.79 (t, J = 7.1 Hz, 3H). |
| Intermediate 19 Ethyl 1-(4-(2- acetamidothiazol-4- yl)phenyl)-5-chloro-3- (3-(3-ethoxy-3- oxopropyl)ureido)-1H- pyrrole-2-carboxylate | 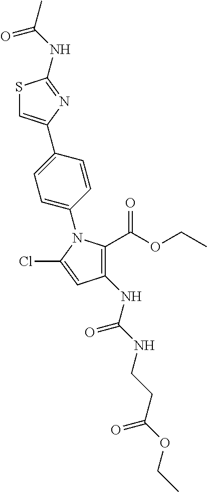 [SMILES: CCOC(=O)CCNC(=O)Nc1cc(Cl)n(-c2ccc(-c3csc(NC(C)=O)n3)cc2)c1C(=O)OCC] | Intermediate 16 and Ethyl 3- isocyanato- proponate | ¹H NMR (400 MHz, DMSO-d6) δ ppm 12.28 (br s, 1H), 8.75 (br s, 1H), 7.98 (d,, 2H), 7.71 (s, 1H), 7.60 (m, 1H), 7.33 (d,, 2H), 6.98 (s, 1H), 4.09 (q, J = 7.1 Hz, 2H), 3.93 (q, J = 7.1 Hz, 2H), 3.29 (s, 3H),, 2.48 (m, 2H), 2.18 (s, 2H),, 1.20 (t, J = 7.1 Hz, 3H), 0.79 (t, J = 7.1 Hz, 3H). |
| Intermediate 20 Ethyl 1-{4-[2- (acetylamino)-1,3- thiazol-4-yl]phenyl}-5- chloro-3-({[(2- fluorophenyl)amino] carbonyl}amino)-1H- pyrrole-2-carboxylate | 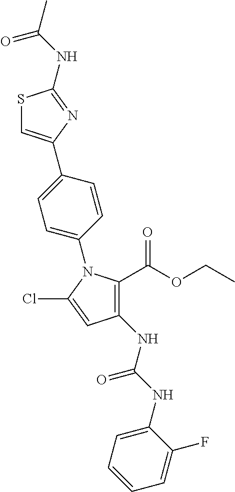 [SMILES: CCOC(=O)c1c(NC(=O)Nc2ccccc2F)cc(Cl)n1-c1ccc(-c2csc(NC(C)=O)n2)cc1] | Intermediate 16 and 1- Fluoro-2- isocyanato- benzene | LCMS: (M + H)⁺ = 542; Rt = 3.61 min. |
| Intermediate 21 Ethyl 1-{4-[2- (acetylamino)-1,3- thiazol-4-yl]phenyl}-5- chloro-3-[({[2-methyl- 4-(methyloxy) phenyl]amino} carbonyl)amino]- 1H-pyrrole-2- carboxylate | 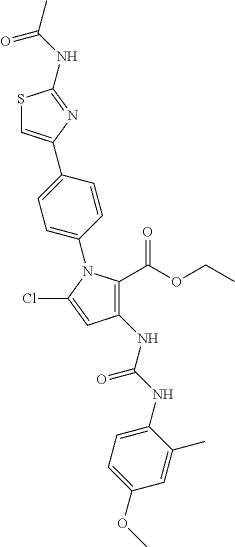 [SMILES: CCOC(=O)c1c(NC(=O)Nc2ccc(OC)cc2C)cc(Cl)n1-c1ccc(-c2csc(NC(C)=O)n2)cc1] | Intermediate 16 and 1- Isocyanato- 2-methyl-4- (methyloxy) benzene | LCMS: (M + H)⁺ = 568; Rt = 3.42 min. |
| Intermediate 22 Ethyl 1-{4-[2- (acetylamino)-1,3- thiazol-4-yl]phenyl}-3- [({[3,5- bis(methyloxy)phenyl] amino}carbonyl) amino]-5-chloro-1H- pyrrole-2-carboxylate | 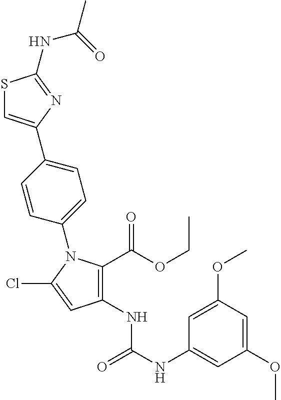 [SMILES: CCOC(=O)c1c(NC(=O)Nc2cc(OC)cc(OC)c2)cc(Cl)n1-c1ccc(-c2csc(NC(C)=O)n2)cc1] | Intermediate 16 and 1- Isocyanato- 3,5- bis(methyl- oxy)benzene | LCMS: (M + H)⁺ = 584; Rt = 3.57 min. |
| Intermediate 23 Ethyl 1-{4-[2- (acetylamino)-1,3- thiazol-4-yl]phenyl}-3- [({[2,5- bis(methyloxy)phenyl] amino}carbonyl) amino]-5-chloro- 1H-pyrrole- 2-carboxylate | 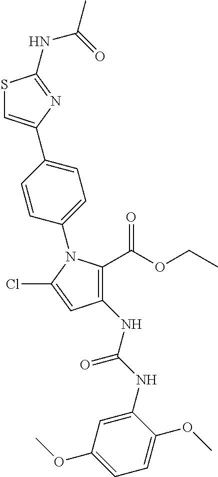 [SMILES: CCOC(=O)c1c(NC(=O)Nc2cc(OC)ccc2OC)cc(Cl)n1-c1ccc(-c2csc(NC(C)=O)n2)cc1] | Intermediate 16 and 2- Isocyanato- 1,4- dimethoxy- benzene | LCMS: (M + H)⁺ = 584; Rt = 3.84 min. |
| Intermediate 24 Ethyl 1-{4-[2- (acetylamino)-1,3- thiazol-4-yl]phenyl}-5- chloro-3-[({[3- (methylthio)phenyl] amino}carbonyl) amino]-1H-pyrrole-2- carboxylate | 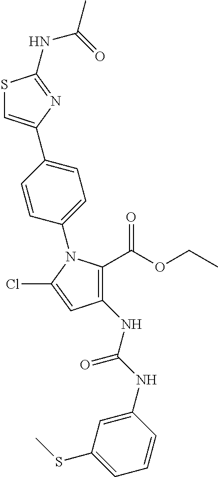 [SMILES: CCOC(=O)c1c(NC(=O)Nc2cccc(SC)c2)cc(Cl)n1-c1ccc(-c2csc(NC(C)=O)n2)cc1] | Intermediate 16 and 1- Isocyanato- 3- (methylthio) benzene | LCMS: (M + H)⁺ = 570; Rt = 4.04 min. |
| Intermediate 25 Ethyl 1-{4-[2- (acetylamino)-1,3- thiazol-4-yl]phenyl}-3- {[(1,3-benzodioxol-5- ylamino)carbonyl] amino}-5-chloro-1H- pyrrole-2-carboxylate | 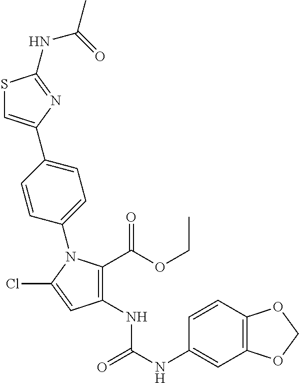 [SMILES: CCOC(=O)c1c(NC(=O)Nc2ccc3c(c2)OCO3)cc(Cl)n1-c1ccc(-c2csc(NC(C)=O)n2)cc1] | Intermediate 16 and 5- Isocyanato- 1,3-benzo- dioxole | LCMS: (M + H)⁺ = 568; Rt = 3.72 min. |
| Intermediate 26 Ethyl 1-{4-[2- (acetylamino)-1,3- thiazol-4-yl]phenyl}-5- chloro-3-{[(3- pyridinylamino) carbonyl]amino}- 1H-pyrrole- 2-carboxylate | 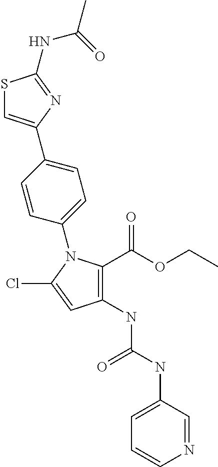 [SMILES: CCOC(=O)c1c(NC(=O)Nc2cccnc2)cc(Cl)n1-c1ccc(-c2csc(NC(C)=O)n2)cc1] | Intermediate 16 and 3- Isocyanato- pyridine | LCMS: (M + H)⁺ = 525; Rt = 3.23 min. |
| Intermediate 27 Ethyl 1-{4-[2- (acetylamino)-1,3- thiazol-4-yl]phenyl}-5- chloro-3-[({[2- (methyloxy)phenyl] amino}carbonyl) amino]-1H-pyrrole-2- carboxylate | 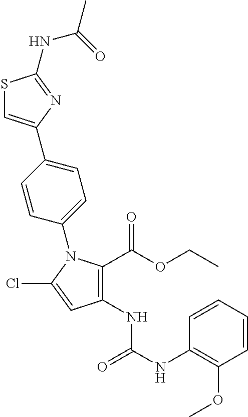 [SMILES: CCOC(=O)c1c(NC(=O)Nc2ccccc2OC)cc(Cl)n1-c1ccc(-c2csc(NC(C)=O)n2)cc1] | Intermediate 16 and 1- Isocyanato- 2- (methyloxy) benzene | LCMS: (M + H)⁺ = 554; Rt = 3.64 min. |

### Intermediate 28

### N-[4-(4-{6-Chloro-3-[3-(methyloxy)phenyl]-2,4-dioxo-1,2,3,4-tetrahydro-5H-pyrrolo[3,2-d]pyrimidin-5-yl}phenyl)-1,3-thiazol-2-yl]acetamide

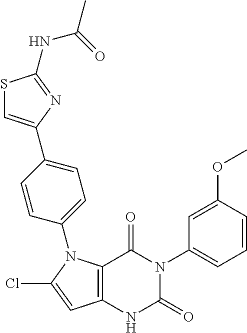 [SMILES: COc1cccc(-n2c(=O)[nH]c3cc(Cl)n(-c4ccc(-c5csc(NC(C)=O)n5)cc4)c3c2=O)c1]

A solution of EtONa, freshly prepared from sodium (1.145 g, 49.8 mmol) and ethanol (20 mL) was added dropwise to a solution of ethyl 1-{4-[2-(acetylamino)-1,3-thiazol-4-yl]phenyl}-5-chloro-3-[({[3-(methyloxy)phenyl]amino}carbonyl)amino]-1H-pyrrole-2-carboxylate (Intermediate 17) (9.2 g, 16.61 mmol) in ethanol (60 mL) at RT. The reaction was stirred at 60° C. for 2 h before being concentrated in vacuo. The residue was taken up in water and 1N HCl was added until pH ˜2. The precipitate was filtered and washed with iPr₂O (10 mL). The product was then dried under reduced pressure to give the title compound (8 g, 15.75 mmol, 95% yield) as a cream solid. LCMS: (M+H)⁺=508; Rt=2.81 min.

Intermediates 29 to 34 were prepared by methods analogous to that described for intermediate 28. For Intermediates 29 30 and 34, MeONa was used instead of EtONa.

TABLE 2

| Intermediate | Formula | From | Physical Data |
|---|---|---|---|
| Intermediate 29 N-[4-(4-{3-[3,5- Bis(methyloxy) phenyl]-6-chloro- 2,4-dioxo- 1,2,3,4-tetrahydro- 5H-pyrrolo[3,2- d]pyrimidin-5- yl}phenyl)-1,3- thiazol-2- yl]acetamide | 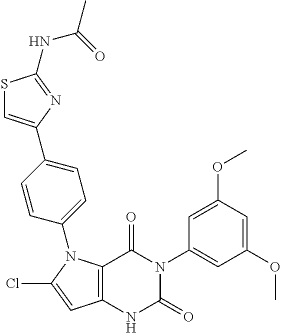 [SMILES: COc1cc(OC)cc(-n2c(=O)[nH]c3cc(Cl)n(-c4ccc(-c5csc(NC(C)=O)n5)cc4)c3c2=O)c1] | Ethyl 1-{4-[2- (acetylamino)-1,3- thiazol-4- yl]phenyl}-3-[({[3,5- bis(methyloxy) phenyl]amino} carbonyl) amino]-5-chloro- 1H-pyrrole-2- carboxylate (Intermediate 22) | LCMS: (M + H)⁺ = 538; Rt = 3.04 min. |
| Intermediate 30 N-(4-{4-[3-(1,3- Benzodioxol-5-yl)-6- chloro-2,4-dioxo- 1,2,3,4-tetrahydro- 5H-pyrrolo[3,2- d]pyrimidin-5- yl]phenyl}-1,3- thiazol-2- yl)acetamide | 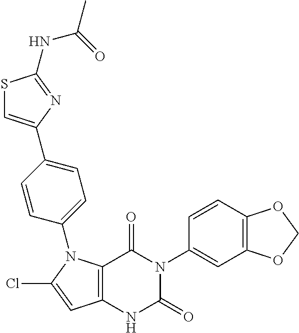 [SMILES: CC(=O)Nc1nc(-c2ccc(-n3c(Cl)cc4[nH]c(=O)n(-c5ccc6c(c5)OCO6)c(=O)c43)cc2)cs1] | Ethyl 1-{4-[2- (acetylamino)-1,3- thiazol-4- yl]phenyl}-3-{[(1,3- benzodioxol-5- ylamino)carbonyl] amino}-5-chloro- 1H-pyrrole-2- carboxylate (Intermediate 25) | LCMS: (M − H)⁺ = 520; Rt = 3.03 min. |
| Intermediate 31 N-(4-{4-[6-Chloro- 2,4-dioxo-3-(3- pyridinyl)-1,2,3,4- tetrahydro-5H- pyrrolo[3,2- d]pyrimidin-5- yl]phenyl}-1,3- thiazol-2- yl)acetamide, hydrochloride | 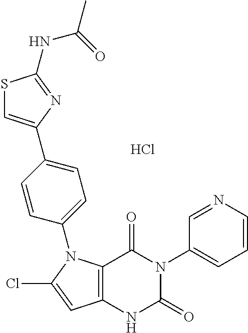 [SMILES: CC(=O)Nc1nc(-c2ccc(-n3c(Cl)cc4[nH]c(=O)n(-c5cccnc5)c(=O)c43)cc2)cs1 ; Cl] | Ethyl 1-{4-[2- (acetylamino)-1,3- thiazol-4- yl]phenyl}-5-chloro- 3-{[(3- pyridinylamino) carbonyl]amino}- 1H-pyrrole-2- carboxylate (Intermediate 26) | LCMS: (M + H)⁺ = 479; Rt = 2.72 min. |
| Intermediate 32 N-(4-(4-(6-Chloro-3- (3-methoxy-2- methylphenyl)-2,4- dioxo-3,4-dihydro- 1H-pyrrolo[3,2- d]pyrimidin-5(2H)- yl)phenyl)thiazol-2- yl)acetamide | 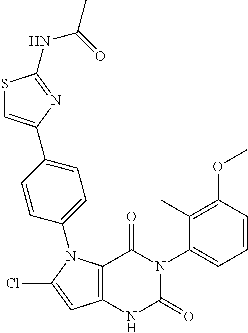 [SMILES: COc1cccc(-n2c(=O)[nH]c3cc(Cl)n(-c4ccc(-c5csc(NC(C)=O)n5)cc4)c3c2=O)c1C] | Ethyl 1-{4-[2- (acetylamino)-1,3- thiazol-4- yl]phenyl}-5-chloro- 3- [({[2-methyl-3- (methyloxy)phenyl] amino}carbonyl) amino]-1H- pyrrole-2- carboxylate (Intermediate 40) | LCMS: (M + H)⁺ = 522; Rt = 2.88 min. |
| Intermediate 33 N-(4-(4-(6-Chloro-3- (2-methoxyphenyl)- 2,4-dioxo-3,4- dihydro-1H- pyrrolo[3,2- d]pyrimidin-5(2H)- yl)phenyl)thiazol-2- yl)acetamide | 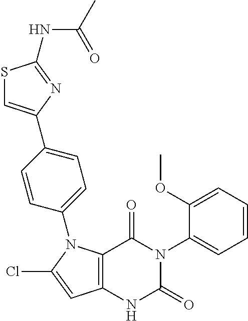 [SMILES: COc1ccccc1-n1c(=O)[nH]c2cc(Cl)n(-c3ccc(-c4csc(NC(C)=O)n4)cc3)c2c1=O] | Ethyl 1-{4-[2- (acetylamino)-1,3- thiazol-4- yl]phenyl}-5-chloro- 3-[({[2- (methyloxy)phenyl] amino}carbonyl) amino]-1H- pyrrole-2- carboxylate (Intermediate 27) | LCMS: (M + H)⁺ = 508; Rt = 2.73 min. |
| Intermediate 34 N-(4-(4-(6-Chloro- 2,4-dioxo-3-(m-tolyl)- 3,4-dihydro-1H- pyrrolo[3,2- d]pyrimidin-5(2H)- yl)phenyl)thiazol-2- yl)acetamide | 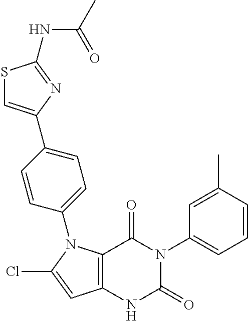 [SMILES: CC(=O)Nc1nc(-c2ccc(-n3c(Cl)cc4[nH]c(=O)n(-c5cccc(C)c5)c(=O)c43)cc2)cs1] | Ethyl 1-(4-(2- acetamidothiazol- 4- yl)phenyl)-5- chloro-3-(3-(m- tolyl)ureido)-1H- pyrrole-2- carboxylate (Intermediate 41) | LCMS: (M + H)⁺ = 492; Rt = 2.87 min. |

### Intermediate 35

### 3-(5-(4-(2-Acetamidothiazol-4-yl)phenyl)-6-chloro-2,4-dioxo-1H-pyrrolo[3,2-d]pyrimidin-3(2H,4H,5H)-yl)benzoic acid

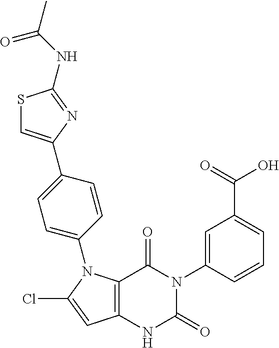 [SMILES: CC(=O)Nc1nc(-c2ccc(-n3c(Cl)cc4[nH]c(=O)n(-c5cccc(C(=O)O)c5)c(=O)c43)cc2)cs1]

To a solution of ethyl 1-(4-(2-acetamidothiazol-4-yl)phenyl)-5-chloro-3-(3-(3-(methoxycarbonyl)phenyl)ureido)-1H-pyrrole-2-carboxylate Intermediate 18 (348 mg, 0.6 mmol) in a THF (12 mL) was added a solution of potassium tertbutoxide (134 mg, 1.19 mmol) in EtOH (3 mL). The reaction mixture was stirred at 70° C. for 30 min before being quenched with 1N HCl. Water was added and the aqueous layer was extracted with EtOAc (2×50 mL). The organic layer was washed with 1N NaOH and the aqueous layer was acidified with 3N HCl then extracted with EtOAc (2×50 mL). The organic layer was dried over sodium sulfate, filtered and concentrated under reduced pressure. The crude product was purified by flash chromatography on silica gel using DCM/THF (6/4) as eluant to give the title compound (195 mg, 63% yield). ¹H NMR (400 MHz, DMSO-d6) δ ppm 7.97 (d, J=8.6 Hz, 2H), 7.85 (m, 1H), 7.70 (s, 1H), 7.67 (brs., 1H), 7.45 (d, J=8.6 Hz, 2H), 7.39 (brs 1H), 6.32 (s, 1H), 5.76 (s, 1H), 3.30 (s, 3H).

Intermediate 36 was prepared by methods analogous to that described for intermediate 35 34.

TABLE 3

| Intermediate | Formula | From | Physical Data |
|---|---|---|---|
| Intermediate 36 3-(5-(4-(2- Acetamidothiazol-4- yl)phenyl)-6-chloro- 2,4-dioxo-1H- pyrrolo[3,2- d]pyrimidin- 3(2H,4H,5H)- yl)propanoic acid | 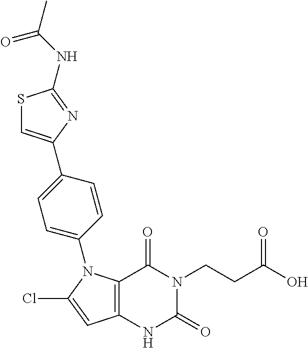 [SMILES: CC(=O)Nc1nc(-c2ccc(-n3c(Cl)cc4[nH]c(=O)n(CCC(=O)O)c(=O)c43)cc2)cs1] | Ethyl 1-(4-(2- acetamidothiazol- 4-yl)phenyl)- 5-chloro-3-(3- (3-ethoxy-3- oxopropyl)ureido)- 1H- pyrrole-2- carboxylate (Intermediate 19) | ¹H NMR (400 MHz, DMSO-d6) δ ppm 12.28 (s, 2H), 11.41 (s, 1H), 8.00 (d, J = 8.1 Hz, 2H), 7.74 (s, 1H), 7.41 (d, J = 8.1 Hz, 2H), 6.24 (s, 1H), 3.97 (m, 2H), 3.32 (s, 3H), 2.42 (m, 2H). |

### Intermediate 37

### Ethyl 1-{4-[2-(acetylamino)-1,3-thiazol-4-yl]phenyl}-5-chloro-3-({[(4-nitrophenyl)oxy]carbonyl}amino)-1H-pyrrole-2-carboxylate

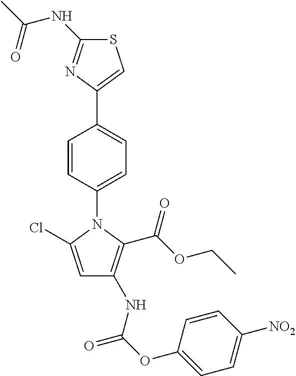 [SMILES: CCOC(=O)c1c(NC(=O)Oc2ccc([N+](=O)[O-])cc2)cc(Cl)n1-c1ccc(-c2csc(NC(C)=O)n2)cc1]

To a solution of ethyl 1-{4-[2-(acetylamino)-1,3-thiazol-4-yl]phenyl}-3-amino-5-chloro-1H-pyrrole-2-carboxylate (Intermediate 16) (405 mg, 1 mmol) in DCM (10 mL) at 0° C. was added a solution of 4-nitrophenyl 4-nitrophenyl carbonochloridate (242 mg, 1.2 mmol) in DCM (2 mL) and the reaction mixture was then stirred at room temperature overnight. DCM was evaporated under reduced pressure and the solid obtained was triturated from diisopropyl ether, filtered and dried to give the title compound (550 mg, 0.965 mmol, 96% yield) as a white solid. LCMS: (M+H)⁺=570; Rt=3.91 min.

Intermediate 38 was prepared by methods analogous to that described for intermediate 37 starting from Intermediate 7.

TABLE 4

| Intermediate | Formula | From | Physical Data |
|---|---|---|---|
| Intermediate 38 ethyl 1-(4-(6- acetamidopyridin-2- yl)phenyl)-5-chloro- 3-(((4- nitrophenoxy) carbonyl)amino)- 1H-pyrrole-2- carboxylate | 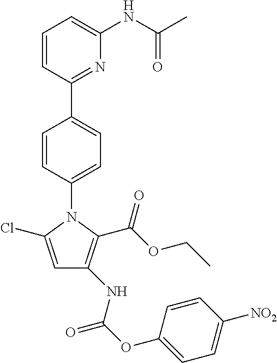 [SMILES: CCOC(=O)c1c(NC(=O)Oc2ccc([N+](=O)[O-])cc2)cc(Cl)n1-c1ccc(-c2cccc(NC(C)=O)n2)cc1] | Ethyl 1-{4-[6- (acetylamino)-2- pyridinyl] phenyl}- 3-amino-5- chloro-1H- pyrrole-2- carboxylate (Intermediate 7) | LCMS: (M + H)⁺ = 564; Rt = 3.93 min. |

### Intermediate 39

### Ethyl 1-{4-[2-(acetylamino)-1,3-thiazol-4-yl]phenyl}-5-chloro-3-[({[4-(methyloxy)phenyl]amino}carbonyl)amino]-1H-pyrrole-2-carboxylate

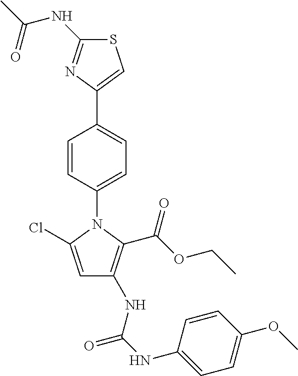 [SMILES: CCOC(=O)c1c(NC(=O)Nc2ccc(OC)cc2)cc(Cl)n1-c1ccc(-c2csc(NC(C)=O)n2)cc1]

To a suspension of ethyl 1-{4-[2-(acetylamino)-1,3-thiazol-4-yl]phenyl}-5-chloro-3-({[(4-nitrophenyl)oxy]carbonyl}amino)-1H-pyrrole-2-carboxylate (Intermediate 37) (250 mg, 0.439 mmol) in THF (10 mL) at RT was added 4-methoxyaniline (59.4 mg, 0.482 mmol). The reaction vessel was sealed and heated in microwave conditions using initial 150W to 120° C. for 40 min. THF was evaporated under reduced pressure and the residue was triturated in diisopropyl ether and filtered to give the title compound (110 mg, 0.199 mmol, 45.3% yield) as a white solid. LCMS: (M+H)⁺=554; Rt=3.57 min.

Intermediate 40 and 41 were prepared by methods analogous to that described for intermediate 39 starting from Intermediate 37 using the appropriate amine.

TABLE 5

| Intermediate | Formula | From | Physical Data |
|---|---|---|---|
| Intermediate 40 Ethyl 1-{4-[2- (acetylamino)-1,3- thiazol-4-yl]phenyl}- 5-chloro-3-[({[2- methyl-3- (methyloxy)phenyl] amino}carbonyl) amino]-1H-pyrrole-2- carboxylate | 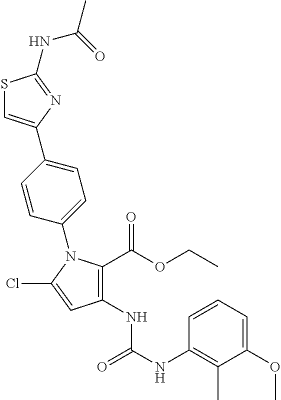 [SMILES: CCOC(=O)c1c(NC(=O)Nc2cccc(OC)c2C)cc(Cl)n1-c1ccc(-c2csc(NC(C)=O)n2)cc1] | Intermediate 37 and 2- methyl- 3methyloxy- aniline | LCMS: (M + H)⁺ = 568; Rt = 3.54 min. |
| Intermediate 41 Ethyl 1-(4-(2- acetamidothiazol-4- yl)phenyl)-5-chloro- 3-(3-(m-tolyl)ureido)- 1H-pyrrole-2- carboxylate | 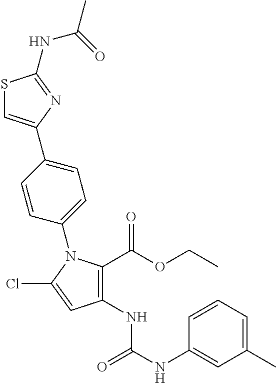 [SMILES: CCOC(=O)c1c(NC(=O)Nc2cccc(C)c2)cc(Cl)n1-c1ccc(-c2csc(NC(C)=O)n2)cc1] | Intermediate 37 and m- toluidine | LCMS: (M + H)⁺ = 538 Rt = 3.72 min. |

Intermediate 42 was prepared by methods analogous to that described for intermediate 39 starting from Intermediate 38 using the appropriate amine.

TABLE 6

| Intermediate | Formula | From | Physical Data |
|---|---|---|---|
| Intermediate 42 Ethyl 1-(4-(6- acetamidopyridin-2- yl)phenyl)-5-chloro- 3-(3-(3-methoxy-2- methylphenyl) ureido)-1H- pyrrole-2- carboxylate | 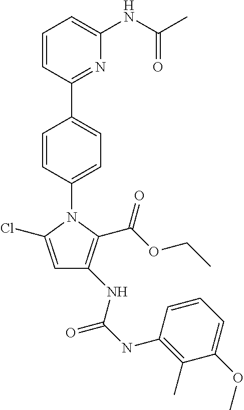 [SMILES: CCOC(=O)c1c(NC(=O)Nc2cccc(OC)c2C)cc(Cl)n1-c1ccc(-c2cccc(NC(C)=O)n2)cc1] | Intermediate 38 and 3-methoxy-2- methylaniline | LCMS: (M + H)⁺ = 562 Rt = 3.64 min. |

### Example 1

### 5-(4-(6-Aminopyridin-2-yl)phenyl)-6-chloro-3-(3-methoxyphenyl)-1H-pyrrolo[3,2-d]pyrimidine-2,4(3H,5H)-dione hydrochloride

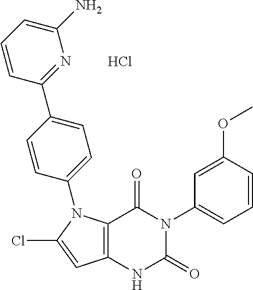 [SMILES: COc1cccc(-n2c(=O)[nH]c3cc(Cl)n(-c4ccc(-c5cccc(N)n5)cc4)c3c2=O)c1 ; Cl]

A solution of MeONa, freshly prepared from sodium (40.4 mg, 1.757 mmol) and methanol (10 mL) was added dropwise at RT to a solution of ethyl 1-{4-[6-(acetylamino)-2-pyridinyl]phenyl}-5-chloro-3-[({[3-(methyloxy)phenyl]amino}carbonyl)amino]-1H-pyrrole-2-carboxylate (Intermediate 8) (321 mg, 0.586 mmol) in methanol (15 mL). The reaction was heated at 80° C. overnight before being concentrated in vacuo. The residue was taken in conc. HCl and stirred 1 h at 60° C. The precipitate was filtered off and the filtrate was concentrated under reduced pressure. The residue was dissolved in MeOH and precipitated with iPr₂O, washed with iPr₂O. The solid was triturated from hot MeCN and filtered and dried to give. title compound (100 mg, 0.191 mmol, 32.7% yield) as a white solid. LCMS: (M+H)⁺=460; Rt=2.81 min. ¹H NMR (300 MHz, DMSO-d₆) δ ppm 11.57 (s, 1H), 8.02 (d, J=8.4 Hz, 2H), 7.94 (m, 1H), 7.61 (d, J=8.4 Hz, 2H), 7.40-7.15 (m, 2H), 7.08-6.88 (m, 2H), 6.88-6.66 (m, 2H), 6.35 (s, 1H), 3.72 (s, 3H).

Examples 2 were prepared by methods analogous to that described for Example 1 starting from intermediate 42 where MeONa was used instead of EtONa

TABLE 7

| Example | Structure | From | Physical Data |
|---|---|---|---|
| Example 2 5-(4-(6- Aminopyridin-2- yl)phenyl)-6-chloro- 3-(3-methoxy-2- methylphenyl)-1H- pyrrolo[3,2- d]pyrimidine- 2,4(3H,5H)-dione hydrochloride | 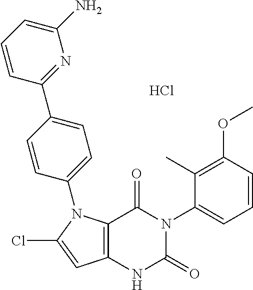 [SMILES: COc1cccc(-n2c(=O)[nH]c3cc(Cl)n(-c4ccc(-c5cccc(N)n5)cc4)c3c2=O)c1C ; Cl] | ethyl 1-(4-(6- acetamidopyridin- 2- yl)phenyl)-5- chloro-3-(3-(3- methoxy-2- methylphenyl) ureido)-1H- pyrrole-2- carboxylate (Intermediate 42) | LCMS (M + H)⁺ = 474 Rt = 2.88 min. HRMS: calculated for C₂₅H₂₀ClN₅O₃ (M + H)⁺: 474.1333; found: 474.1326. |

### Example 3

### 5-[4-(2-Amino-1,3-thiazol-4-yl)phenyl]-6-chloro-3-[3-(methyloxy)phenyl]-1H-pyrrolo[3,2-d]pyrimidine-2,4(3H,5H)-dione hydrochloride

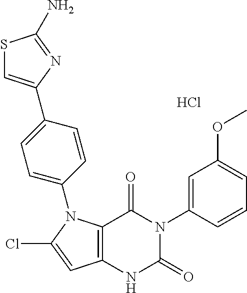 [SMILES: COc1cccc(-n2c(=O)[nH]c3cc(Cl)n(-c4ccc(-c5csc(N)n5)cc4)c3c2=O)c1 ; Cl]

To a solution of N-[4-(4-{6-chloro-3-[3-(methyloxy)phenyl]-2,4-dioxo-1,2,3,4-tetrahydro-5H-pyrrolo[3,2-d]pyrimidin-5-yl}phenyl)-1,3-thiazol-2-yl]acetamide (Intermediate 28) (8 g, 15.75 mmol) in ethanol (50 mL) was added conc. HCl (25.9 mL, 315 mmol) and the reaction mixture was stirred at 80° C. overnight. The solid was filtered and triturated in EtOH and hot filtered. The compound was then dried under reduced pressure to give the title compound (6.22 g, 12.28 mmol, 78% yield) as a white powder. LCMS: (M+H)⁺=466; Rt=2.68 min. HRMS: calculated for C₂₂H₁₇ClN₅O₃S (M+H)⁺: 466.0741. found: 466.0724. ¹H NMR: (DMSO-d⁶, 300 MHz) δ 11.55 (s, 1H), 7.88 (d, J=8.6 Hz, 2H), 7.5 (d, J=8.6 Hz, 2H), 7.31 (m, 2H), 6.94 (m, 1H), 6.79 (m, 2H), 6.33 (s, 1H), 3.73 (s, 3H).

Examples 4 to 10 were prepared by methods analogous to that described for Example 3.

TABLE 8

| Example | Structure | From | Physical Data |
|---|---|---|---|
| Example 4 3-(5-(4-(2- Aminothiazol-4- yl)phenyl)-6-chloro- 2,4-dioxo-1H- pyrrolo[3,2- d]pyrimidin- 3(2H,4H,5H)- yl)propanoic acid hydrochloride | 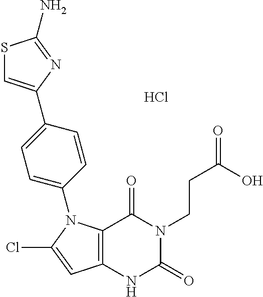 [SMILES: Nc1nc(-c2ccc(-n3c(Cl)cc4[nH]c(=O)n(CCC(=O)O)c(=O)c43)cc2)cs1 ; Cl] | 3-(5-(4-(2- Acetamidothiazol- 4- yl)phenyl)-6- chloro-2,4- dioxo-1H- pyrrolo[3,2- d]pyrimidin- 3(2H,4H,5H)- yl)propanoic acid (Intermediate 36) | LCMS: (M + H)⁺ = 432; Rt = 2.07 min. HRMS: calculated for C₁₈H₁₅ClN₅O₄S (M + H)⁺: 432.0533; found: 432.0511. |
| Example 5 5-[4-(2-amino-1,3- thiazol-4- yl)phenyl]-3-[3,5- bis(methyloxy) phenyl]-6- chloro-1H- pyrrolo[3,2- d]pyrimidine- 2,4(3H,5H)-dione hydrochloride | 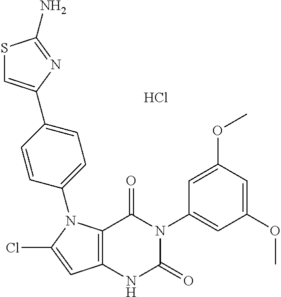 [SMILES: COc1cc(OC)cc(-n2c(=O)[nH]c3cc(Cl)n(-c4ccc(-c5csc(N)n5)cc4)c3c2=O)c1 ; Cl] | N-[4-(4-{3-[3,5- Bis(methyloxy) phenyl]-6- chloro-2,4- dioxo-1,2,3,4- tetrahydro-5H- pyrrolo[3,2- d]pyrimidin-5- yl}phenyl)-1,3- thiazol-2- yl]acetamide (Intermediate 29) | LCMS: (M + H)⁺ = 496; Rt = 2.94 min. HRMS: calculated for C₂₃H₁₉ClN₅O₄S (M + H)⁺: 496.0846; found: 496.0838. |
| Example 6 5-[4-(2-amino-1,3- thiazol-4- yl)phenyl]-3-(1,3- benzodioxol-5-yl)- 6-chloro-1H- pyrrolo[3,2- d]pyrimidine- 2,4(3H,5H)-dione, hydrochloride | 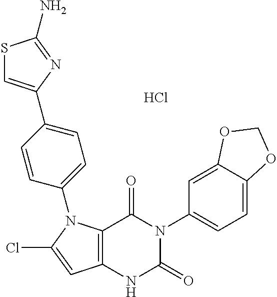 [SMILES: Nc1nc(-c2ccc(-n3c(Cl)cc4[nH]c(=O)n(-c5ccc6c(c5)OCO6)c(=O)c43)cc2)cs1 ; Cl] | N-(4-{4-[3-(1,3- Benzodioxol-5- yl)-6-chloro- 2,4-dioxo- 1,2,3,4- tetrahydro-5H- pyrrolo[3,2- d]pyrimidin-5- yl]phenyl}-1,3- thiazol-2- yl)acetamide (Intermediate 30) | LCMS: (M + H)⁺ = 480; Rt = 2.92 min. HRMS: calculated for C₂₂H₁₅ClN₅O₄S (M + H)⁺: 480.0533; found: 480.0551. |
| Example 7 5-[4-(2-amino-1,3- thiazol-4- yl)phenyl]-6- chloro-3-(3- pyridinyl)-1H- pyrrolo[3,2- d]pyrimidine- 2,4(3H,5H)-dione | 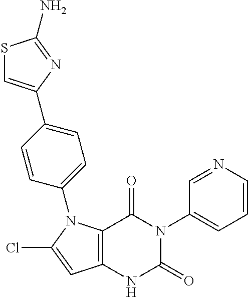 [SMILES: Nc1nc(-c2ccc(-n3c(Cl)cc4[nH]c(=O)n(-c5cccnc5)c(=O)c43)cc2)cs1] | N-(4-{4-[6- Chloro-2,4- dioxo-3-(3- pyridinyl)- 1,2,3,4- tetrahydro-5H- pyrrolo[3,2- d]pyrimidin-5- yl]phenyl}-1,3- thiazol-2- yl)acetamide (Intermediate 31) | LCMS: (M + H)⁺ = 437; Rt = 2.38 min. HRMS: calculated for C₂₀H₁₄ClN₆O₂S (M + H)⁺: 437.0587; found: 437.0598. |
| Example 8 5-(4-(2- Aminothiazol-4- yl)phenyl)-6-chloro- 3-(3-methoxy-2- methylphenyl)-1H- pyrrolo[3,2- d]pyrimidine- 2,4(3H,5H)-dione hydrochloride | 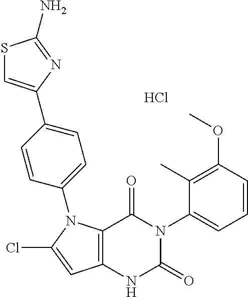 [SMILES: COc1cccc(-n2c(=O)[nH]c3cc(Cl)n(-c4ccc(-c5csc(N)n5)cc4)c3c2=O)c1C ; Cl] | N-(4-(4-(6- Chloro-3-(3- methoxy-2- methylphenyl)- 2,4-dioxo-3,4- dihydro-1H- pyrrolo[3,2- d]pyrimidin- 5(2H)- yl)phenyl) thiazol-2- yl)acetamide (Intermediate 32) | LCMS: (M + H)⁺ = 480; Rt = 2.76 min. HRMS: calculated for C₂₃H₁₉ClN₅O₃S (M + H)⁺: 480.0897; found: 480.0893. |
| Example 9 5-(4-(2- Aminothiazol-4- yl)phenyl)-6-chloro- 3-(2- methoxyphenyl)-1H- pyrrolo[3,2- d]pyrimidine- 2,4(3H,5H)-dione hydrochloride | 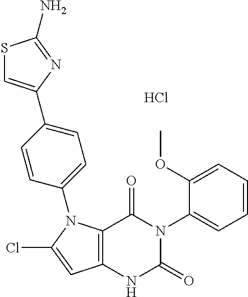 [SMILES: COc1ccccc1-n1c(=O)[nH]c2cc(Cl)n(-c3ccc(-c4csc(N)n4)cc3)c2c1=O ; Cl] | N-(4-(4-(6- Chloro-3-(2- methoxyphenyl)- 2,4-dioxo- 3,4-dihydro- 1H-pyrrolo[3,2- d]pyrimidin- 5(2H)- yl)phenyl) thiazol-2- yl)acetamide (Intermediate 33) | LCMS: (M + H)⁺ = 466; Rt = 2.71 min. HRMS: calculated for C₂₂H₁₇ClN₅O₃S (M + H)⁺: 466.0741; found: 466.0752. |
| Example 10 5-(4-(2- Aminothiazol-4- yl)phenyl)-6-chloro- 3-(m-tolyl)-1H- pyrrolo[3,2- d]pyrimidine- 2,4(3H,5H)-dione hydrochloride | 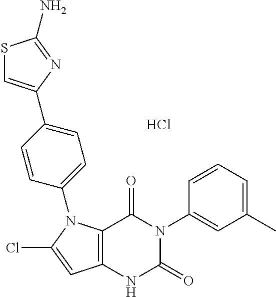 [SMILES: Cc1cccc(-n2c(=O)[nH]c3cc(Cl)n(-c4ccc(-c5csc(N)n5)cc4)c3c2=O)c1 ; Cl] | N-(4-(4-(6- chloro-2,4- dioxo-3-(m- tolyl)-3,4- dihydro-1H- pyrrolo[3,2- d]pyrimidin- 5(2H)- yl)phenyl) thiazol-2- yl)acetamide (intermediate) 34 | LCMS: (M − H)⁺ = 450; Rt = 2.86 min. HRMS: calculated for C₂₂H₁₇ClN₅O₂S (M + H)⁺: 450.0791; found: 450.0777. |

### Example 11

### 3-(5-(4-(2-Aminothiazol-4-yl)phenyl)-6-chloro-2,4-dioxo-1H-pyrrolo[3,2-d]pyrimidin-3(2H,4H,5H)-yl)benzoic acid hydrochloride

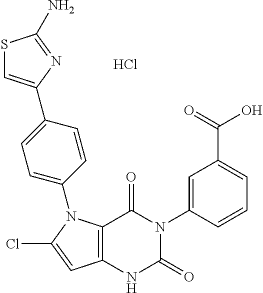 [SMILES: Nc1nc(-c2ccc(-n3c(Cl)cc4[nH]c(=O)n(-c5cccc(C(=O)O)c5)c(=O)c43)cc2)cs1 ; Cl]

To a solution of 3-(5-(4-(2-acetamidothiazol-4-yl)phenyl)-6-chloro-2,4-dioxo-1H-pyrrolo[3,2-d]pyrimidin-3(2H,4H,5H)-yl)benzoic acid (Intermediate 35) (160 mg, 0.3 mmol) in a MeOH (64 mL)/water (16 mL) mixture was added 12N HCl (1.6 mL, 1.9 mmol). The reaction mixture was stirred at reflux for 24 h before being concentrated to dryness. The crude product contained a mixture of the desired product and its corresponding methyl ester. To a solution of the previous compounds (assumed 158 mg, 0.3 mmol) in MeOH (10 mL) was added a solution of LiOH (89 mg, 2.1 mmol) in water (2 mL). The reaction mixture was stirred at 50° C. for 45 h before being concentrated. Water was added and pH adjusted to 1 with 3N HCl. The resulting precipitate was filtered, washed with cold water and dried to give the title compound as a grey solid. (100 mg, 65% yield) as a. ¹H NMR (400 MHz, DMSO-d6) δ ppm 11.61 (s, 1H), 7.92 (d, 1H), 7.85 (d, 2H), 7.75 (s, 1H), 7.54 (m, 1H), 7.51-7.40 (m, 3H), 7.22 (s, 1H), 6.33 (s, 1H). LCMS: (M+H)⁺=480.

### Example 12

### 5-[4-(2-Amino-1,3-thiazol-4-yl)phenyl]-6-chloro-3-(2-fluorophenyl)-1H-pyrrolo[3,2-d]pyrimidine-2,4(3H,5H)-dione

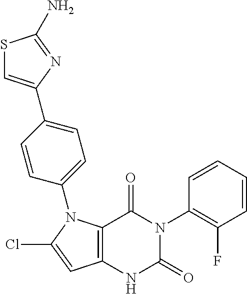 [SMILES: Nc1nc(-c2ccc(-n3c(Cl)cc4[nH]c(=O)n(-c5ccccc5F)c(=O)c43)cc2)cs1]

To a solution of ethyl 1-{4-[2-(acetylamino)-1,3-thiazol-4-yl]phenyl}-5-chloro-3-({[(2-fluorophenyl)amino]carbonyl}amino)-1H-pyrrole-2-carboxylate (Intermediate 20) (340 mg, 0.627 mmol) in DCM (20 mL) was added dropwise a solution of MeONa, freshly prepared from sodium (28.8 mg, 1.255 mmol) and methanol (20 mL). The reaction mixture was stirred at RT overnight before being concentrated under reduced pressure then diluted with MeOH (10 mL) and water (10 mL). KOH (176 mg, 3.14 mmol) was added then the reaction vessel was sealed and heated in microwave conditions to 130° C. for 20 min. The reaction mixture was extracted with DCM (2×30 mL). The organic layer was washed with brine, dried over anhydrous Na₂SO₄, filtered and concentrated in vacuo. The sample was loaded on 25 g silica (Si) column then the purification was carried out using a DCM/MeOH 100/0 to 70/30 gradient. The appropriate fractions were combined and concentrated in vacuo to give the required product (95 mg, 33% yield) as a light yellow powder. LCMS: (M+H)⁺=454; Rt=2.74 min. HRMS: calculated for C₂₁H₁₂ClFN₅O₂S (M−H)⁻: 452.0385. found: 452.0381.

Examples 13 to 17 were prepared by methods analogous to that described for Example 12. For example 14, 10N HCl was used instead of KOH.

TABLE 6

| Example | Structure | From | Physical Data |
|---|---|---|---|
| Example 13 5-(4-(2- Aminothiazol-4- yl)phenyl)-6-chloro- 3-(4-methoxy-2- methylphenyl)-1H- pyrrolo[3,2- d]pyrimidine- 2,4(3H,5H)-dione | 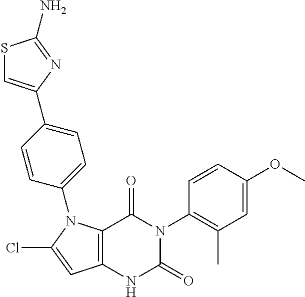 [SMILES: COc1ccc(-n2c(=O)[nH]c3cc(Cl)n(-c4ccc(-c5csc(N)n5)cc4)c3c2=O)c(C)c1] | Ethyl 1-{4-[2- (acetylamino)- 1,3-thiazol-4- yl]phenyl}-5- chloro-3-[({[2- methyl-4- (methyloxy) phenyl]amino} carbonyl) amino]- 1H-pyrrole-2- carboxylate (Intermediate 21) | LCMS: (M + H)⁺ = 480; Rt = 2.71 min. HRMS: calculated for C₂₃H₁₇ClN₅O₃S (M − H)⁻: 478.0741; found: 478.0724. |
| Example 14 5-[4-(2-Amino-1,3- thiazol-4- yl)phenyl]-6- chloro-3-[4- (methyloxy) phenyl]-1H- pyrrolo[3,2- d]pyrimidine- 2,4(3H,5H)-dione hydrochloride | 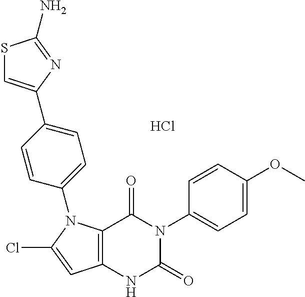 [SMILES: COc1ccc(-n2c(=O)[nH]c3cc(Cl)n(-c4ccc(-c5csc(N)n5)cc4)c3c2=O)cc1 ; Cl] | Ethyl 1-{4-[2- (acetylamino)- 1,3-thiazol-4- yl]phenyl}-5- chloro-3-[({[4- (methyloxy) phenyl]amino} carbonyl)amino]- 1H-pyrrole-2- carboxylate (Intermediate 39) | LCMS: (M + H)⁺ = 466; Rt = 2.86 min. HRMS: calculated for C₂₂H₁₇ClN₅O₃S (M + H)⁻: 466.0741; found: 466.0754. |
| Example 15 5-[4-(2-Amino-1,3- thiazol-4- yl)phenyl]-3-[2,5- bis(methyloxy) phenyl]-6-chloro- 1H-pyrrolo[3,2- d]pyrimidine- 2,4(3H,5H)-dione | 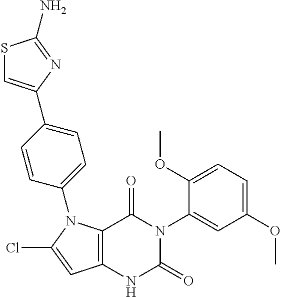 [SMILES: COc1ccc(OC)c(-n2c(=O)[nH]c3cc(Cl)n(-c4ccc(-c5csc(N)n5)cc4)c3c2=O)c1] | Ethyl 1-{4-[2- (acetylamino)- 1,3-thiazol-4- yl]phenyl}-3- [({[2,5- bis(methyloxy) phenyl]amino} carbonyl)amino]- 5-chloro-1H- pyrrole-2- carboxylate (Intermediate 23) | LCMS: (M + H)⁺ = 496; Rt = 2.93 min. HRMS: calculated for C₂₃H₁₇ClN₅O₄S(M − H)⁻: 494.0690; found: 494.0681. |
| Example 16 5-[4-(2-Amino-1,3- thiazol-4-yl)phenyl]- 6-chloro-3-[3- (methylthio)phenyl]- 1H-pyrrolo[3,2- d]pyrimidine- 2,4(3H,5H)-dione | 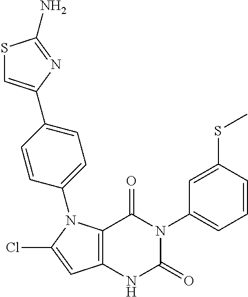 [SMILES: CSc1cccc(-n2c(=O)[nH]c3cc(Cl)n(-c4ccc(-c5csc(N)n5)cc4)c3c2=O)c1] | Ethyl 1-{4-[2- (acetylamino)- 1,3-thiazol-4- yl]phenyl}-5- chloro-3-[({[3- (methylthio) phenyl]amino} carbonyl)amino]- 1H-pyrrole-2- carboxylate (Intermediate 24) | LCMS: (M + H)⁺ = 482; Rt = 3.07 min. HRMS: calculated for C₂₂H₁₅ClN₅O₂S₂ (M − H⁺ 480.0356; found: 480.0406. |

### Example 17

### 5-(4-(2-Aminothiazol-4-yl)phenyl)-6-chloro-3-(3-hydroxyphenyl)-1H-pyrrolo[3,2-d]pyrimidine-2,4(3H,5H)-dione, Hydrochloride

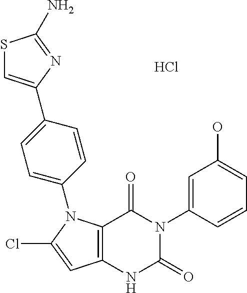 [SMILES: Nc1nc(-c2ccc(-n3c(Cl)cc4[nH]c(=O)n(-c5cccc(O)c5)c(=O)c43)cc2)cs1 ; Cl]

To a solution of 5-(4-(2-aminothiazol-4-yl)phenyl)-6-chloro-3-(3-methoxyphenyl)-1H-pyrrolo[3,2-d]pyrimidine-2,4(3H,5H)-dione hydrochloride (example 3) (130 mg, 0.259 mmol) DCM (5 mL) was slowly added BBr₃ (0.776 mL, 0.776 mmol) and the reaction mixture was stirred at RT overnight. Water was added and product was extracted with DCM. Solvent was removed under reduced pressure, then it was dissolved in water and HCl 1N was added. The solid was filtered and tritured in hot MeCN. The solid was purified by HPLC to give the title compound (4 mg, 7.37 μmol, 2.85% yield) as a white solid.

LCMS: (M+H)⁺=452; Rt=2.44 min.

HRMS: calculated for C₂₁H₁₅ClN₅O₃S (M+H)⁺: 452.0584. found: 452.0569.

### Biological Assay

### AMPK Enzymatic Assay

Human recombinant AMPK (Invitrogen #PV4673 & #PV4675) is used in a FRET assay format (Z'Lyte—Invitrogen). Assay conditions are as follow: ATP 100 μM, peptide (Invitrogen # PR8650) 2 μM, 1% final DMSO in Z'Lyte kinase buffer. Reaction is initiated by addition of 0.2-0.8 ng of AMPK and incubated for 1-hour @ 30° C. A further 1-hour incubation @ 30° C. with the development reagent (Invitrogen # PR5194) is performed. FRET signal is then measured and converted to “% peptide phosphorylation” according to Z'Lyte given calculation procedure. Evaluation of compounds is carried out using concentration-response curves. Final data are expressed in “% activation” calculating the ratio of “% peptide phosphorylation” between compound-condition and basal-condition. Alternatively pEC200 (−Log(compound concentration leading to a 2-fold AMPK activity increase)) is produced through fitting of the concentration-response curves. All data are means of at least 2 independent experiments.

The compounds or salts of Examples 1 to 17 were tested in the assay described above and gave pEC₅₀ values of greater than or equal to 5.5. In one aspect, the compounds of the invention give a pEC₅₀ value of ≧6.0 when tested in this assay.

For instance, Example compounds 11 and 12 gave an average pEC₅₀ value of 6.2 and 6.1 respectively.

1. A compound of formula (I): 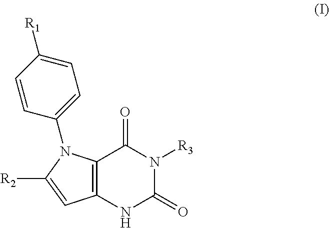
wherein
R¹ represents 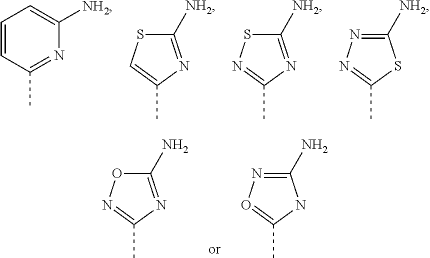
R² represents H, CN, or halogen;
R³ represents
(a) —C₁₋₄alkyl substituted by one or two groups independently selected from: —OH and —CO₂H;
(b) —C₆₋₁₀aryl, or -(5-10 membered heteroaryl), wherein the —C₆₋₁₀aryl or -(5-10 membered heteroaryl) is optionally substituted by one or two groups independently selected from
(i) —C₁₋₄alkyl wherein the alkyl group is unsubstituted or substituted by one or two groups independently selected from: —OH or —CO₂H;
(ii) —OMe;
(iii) —SMe;
(iv) —OH;
(v) —CN;
(vi) —NO₂;
(vii) —CO₂H;
(viii) —C₁₋₄alkylene(C═O)XC₁₋₄alkyl; and
(ix) fluoro;
X represents O or NR⁴; and
R⁴ represents H or —C₁₋₄alkyl;
or a salt thereof;
provided that the compound of formula (I) is not 5-(4-(2-aminothiazol-4-yl)phenyl)-6-chloro-3-(3-fluoro-2-methylphenyl)-1H-pyrrolo[3,2-d]pyrimidine-2,4(3H,5H)-dione.

2. A compound of formula (I) according to claim 1 or a salt thereof, wherein R¹ represents

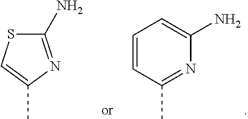

3. A compound of formula (I) according to claim 1 a salt thereof wherein R² represents chloro.

4. A compound of formula (I) according to claim 1 or a salt thereof wherein R³ represents
a) —C₁₋₄alkyl substituted by one or two groups independently selected from: —OH and —CO₂H;
b) C₆₋₁₀aryl optionally substituted with one or two groups independently selected from the group consisting of C₁₋₄alkyl, OMe, SMe, fluoro and CO₂H; or
c) 5-10 membered heteroaryl optionally substituted with one or two groups independently selected from the group consisting of C₁₋₄alkyl, OMe, SMe, fluoro and CO₂H.

5. A compound of formula (I) according to claim 1 or a salt thereof wherein R³ represents —C₁₋₄alkyl substituted by one or two groups independently selected from: —OH and —CO₂H.

6. A compound of formula (I) according to claim 1 or a salt thereof wherein R³ represents C₆₋₁₀aryl optionally substituted with one or two groups independently selected from the group consisting of C₁₋₄alkyl, OMe, SMe, fluoro and CO₂H.

7. A compound of formula (I) according to claim 1 or a salt thereof wherein R³ represents 5-10 membered heteroaryl optionally substituted with one or two groups independently selected from the group consisting of C₁₋₄alkyl, OMe, SMe, fluoro and CO₂H.

8. A compound of formula (I) according to claim 1 selected from the group consisting of:
5-(4-(6-aminopyridin-2-yl)phenyl)-6-chloro-3-(3-methoxyphenyl)-1H-pyrrolo[3,2-d]pyrimidine-2,4(3H,5H)-dione;
5-[4-(2-amino-1,3-thiazol-4-yl)phenyl]-6-chloro-3-[3-(methyloxy)phenyl]-1H-pyrrolo[3,2-d]pyrimidine-2,4(3H,5H)-dione;
3-(5-(4-(2-aminothiazol-4-yl)phenyl)-6-chloro-2,4-dioxo-1H-pyrrolo[3,2-d]pyrimidin-3(2H,4H,5H)-yl)propanoic acid;
5-(4-(2-aminothiazol-4-yl)phenyl)-6-chloro-3-(3,5-dimethoxyphenyl)-1H-pyrrolo[3,2-d]pyrimidine-2,4(3H,5H)-dione;
5-(4-(2-aminothiazol-4-yl)phenyl)-3-(benzo[d][1,3]dioxol-5-yl)-6-chloro-1H-pyrrolo[3,2-d]pyrimidine-2,4(3H,5H)-dione;
5-(4-(2-aminothiazol-4-yl)phenyl)-6-chloro-3-(pyridin-3-yl)-1H-pyrrolo[3,2-d]pyrimidine-2,4(3H,5H)-dione;
5-(4-(2-aminothiazol-4-yl)phenyl)-6-chloro-3-(3-methoxy-2-methylphenyl)-1H-pyrrolo[3,2-d]pyrimidine-2,4(3H,5H)-dione;
5-(4-(2-aminothiazol-4-yl)phenyl)-6-chloro-3-(2-methoxyphenyl)-1H-pyrrolo[3,2-d]pyrimidine-2,4(3H,5H)-dione;
3-(5-(4-(2-aminothiazol-4-yl)phenyl)-6-chloro-2,4-dioxo-1H-pyrrolo[3,2-d]pyrimidin-3(2H,4H,5H)-yl)benzoic acid;
5-(4-(2-aminothiazol-4-yl)phenyl)-6-chloro-3-(2-fluorophenyl)-1H-pyrrolo[3,2-d]pyrimidine-2,4(3H,5H)-dione;
5-(4-(2-aminothiazol-4-yl)phenyl)-6-chloro-3-(4-methoxy-2-methylphenyl)-1H-pyrrolo[3,2-d]pyrimidine-2,4(3H,5H)-dione;
5-(4-(2-aminothiazol-4-yl)phenyl)-6-chloro-3-(4-methoxyphenyl)-1H-pyrrolo[3,2-d]pyrimidine-2,4(3H,5H)-dione;
5-(4-(2-aminothiazol-4-yl)phenyl)-6-chloro-3-(2,5-dimethoxyphenyl)-1H-pyrrolo[3,2-d]pyrimidine-2,4(3H,5H)-dione;
5-(4-(2-aminothiazol-4-yl)phenyl)-6-chloro-3-(3-(methylthio)phenyl)-1H-pyrrolo[3,2-d]pyrimidine-2,4(3H,5H)-dione;
5-(4-(2-aminothiazol-4-yl)phenyl)-6-chloro-3-(m-tolyl)-1H-pyrrolo[3,2-d]pyrimidine-2,4(3H,5H)-dione;
5-(4-(6-aminopyridin-2-yl)phenyl)-6-chloro-3-(3-methoxy-2-methylphenyl)-1H-pyrrolo[3,2-d]pyrimidine-2,4(3H,5H)-dione;
5-(4-(2-aminothiazol-4-yl)phenyl)-6-chloro-3-(3-hydroxyphenyl)-1H-pyrrolo[3,2-d]pyrimidine-2,4(3H,5H)-dione;
and salts thereof.

9. A compound of formula (I) according to claim 1 or a salt thereof wherein the salt is a pharmaceutically acceptable salt.

10. A pharmaceutical composition comprising a) a compound of formula (I) according to claim 9 or pharmaceutically acceptable salt thereof and b) at least one pharmaceutically acceptable carrier.

11-15. (canceled)

16. A method of treating a disease or a condition susceptible to amelioration by an AMPK activator comprising administering to a subject a therapeutically effective amount of a compound for formula (I) according to claim 9 or a pharmaceutically acceptable salt thereof.

17. A method of treating diabetes, metabolic syndrome, atherosclerosis, dyslipidaemia, obesity, hypertension, cerebral ischemia, cognitive defect and/or cancer comprising administering to a subject a therapeutically effective amount of a compound for formula (I) according to claim 9 or a pharmaceutically acceptable salt thereof.
